(19) United States
(12) Patent Application Publication
(10) Pub. No.: US 2007/0026043 A1
(43) Pub. Date: Feb. 1, 2007

(54) Medical devices combined with diblock copolymer compositions

(75) Inventors: Dechi Guan, Vancouver (CA); Richard Whitbourne, Rochester, NY (US); David M. Gravett, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 11/431,427

(22) Filed: May 9, 2006

Related U.S. Application Data
(63) Continuation-in-part of application No. 10/986,450, filed on Nov. 10, 2004.
(63) Continuation-in-part of application No. 10/986,231, filed on Nov. 10, 2004.
(63) Continuation-in-part of application No. 10/986,230, filed on Nov. 10, 2004.
(63) Continuation-in-part of application No. PCT/US05/40512, filed on Nov. 9, 2005.
(60) Provisional application No. 60/523,908, filed on Nov. 20, 2003.
(60) Provisional application No. 60/524,023, filed on Nov. 20, 2003.
(60) Provisional application No. 60/578,471, filed on Jun. 9, 2004.
(60) Provisional application No. 60/582,833, filed on Jun. 24, 2004.
(60) Provisional application No. 60/586,861, filed on Jul. 9, 2004.
(60) Provisional application No. 60/523,908, filed on Nov. 20, 2003.
(60) Provisional application No. 60/524,023, filed on Nov. 20, 2003.
(60) Provisional application No. 60/525,226, filed on Nov. 24, 2003.
(60) Provisional application No. 60/526,541, filed on Dec. 3, 2003.
(60) Provisional application No. 60/578,471, filed on Jun. 9, 2004.
(60) Provisional application No. 60/586,861, filed on Jul. 9, 2004.
(60) Provisional application No. 60/523,908, filed on Nov. 20, 2003.
(60) Provisional application No. 60/524,023, filed on Nov. 20, 2003.
(60) Provisional application No. 60/578,471, filed on Jun. 9, 2004.
(60) Provisional application No. 60/586,861, filed on Jul. 9, 2004.
(60) Provisional application No. 60/625,958, filed on Nov. 9, 2004.

(51) Int. Cl. A61F 2/00; C08F 20/00

(52) U.S. Cl. 424/426; 525/437

(57) ABSTRACT

The present invention provides a medical device combined with a polymeric coating material comprising a bioerodable diblock copolymer and optionally a therapeutic agent.

### CROSS-REFERENCE TO RELATED APPLICATIONS

This application is a continuation-in-part of U.S. patent application Ser. No. 10/986,450 filed Nov. 10, 2004 (currently pending); which claims the benefit under 35 U.S.C. § 119(e) of U.S. Provisional Patent Application Nos. 60/523,908 filed Nov. 20, 2003, 60/524,023 filed Nov. 20, 2003, 60/578,471 filed Jun. 9, 2004, 60/582,833 filed Jun. 24, 2004, and 60/586,861 filed Jul. 9, 2004;

This application is also a continuation-in-part of U.S. patent application Ser. No. 10/986,231 filed Nov. 10, 2004 (currently pending); which claims the benefit under 35 U.S.C. § 119(e) of U.S. Provisional Patent Application Nos. 60/523,908 filed Nov. 20, 2003, 60/524,023 filed Nov. 20, 2003, 60/525,226 filed Nov. 24, 2003, 60/526,541 filed Dec. 3, 2003, 60/578,471 filed Jun. 9, 2004, and 60/586,861 filed Jul. 9, 2004;

This application is also a continuation-in-part of U.S. patent application Ser. No. 10/986,230 filed Nov. 10, 2004 (currently pending); which claims the benefit under 35 U.S.C. § 119(e) of U.S. Provisional Patent Application Nos. 60/523,908 filed Nov. 20, 2003, 60/524,023 filed Nov. 20, 2003, 60/578,471 filed Jun. 9, 2004, and 60/586,861 filed Jul. 9, 2004;

This application is also a continuation-in-part under 35 U.S.C. § 120 of PCT/US2005/040512 filed Nov. 9, 2005; which claims the benefit under 35 U.S.C. § 119(e) of U.S. Provisional Patent Application No. 60/625,958 filed Nov. 9, 2004; where these applications are incorporated herein by reference in their entireties.

### BACKGROUND OF THE INVENTION

1. Field of the Invention

The present invention relates generally to medical devices combined with polymeric compositions, methods of making and using the same, and more specifically, to coated medical devices having improved biocompatibility and efficiency.

2. Description of the Related Art

Advances in the design of medical devices such as catheters, sensors, needles, guide wires, vascular graft, stent graft and stents have greatly improved the quality of medical care. However, the implantation of these devices often brings undesirable complications such as tissue trauma, bacterial infection, blood clots, type I and type II endoleaks, all of which may require ancillary treatments or removal of the devices.

For instance, the clinical function of numerous implantable or insertable devices is dependent upon the device being able to effectively maintain an anatomical, or surgically created, space or passageway. Unfortunately, many devices implanted in the body are subject to a “foreign body” response from the surrounding host tissues. In particular, injury to tubular anatomical structures (such as blood vessels, the gastrointestinal tract, the male and female reproductive tract, the urinary tract, sinuses, spinal nerve root canals, lacrimal ducts, Eustachian tubes, the auditory canal, and the respiratory tract) from surgery and/or injury created by the implantation of medical devices can lead to a well known clinical problem called “stenosis” (or narrowing). Physical injury during an interventional procedure, such as implantation of a stent to open a passageway, results in damage to epithelial lining of the tube and the smooth muscle cells (SMCs) that make up the wall. The damaged cells, particularly SMCs, release cytokines, which recruit inflammatory cells such as macrophages, lymphocytes and neutrophils (i.e., which are some of the known white blood cells) into the area. The white blood cells in turn release a variety of additional cytokines, growth factors, and tissue degrading enzymes that influence the behavior of the constituent cells of the wall (primarily epithelial cells and SMCs). Stimulation of the SMCs induces them to migrate into the inner aspect of the body passageway (often called the “intima”), proliferate and secrete an extracellar matrix—effectively filling all or parts of the lumen with reactive, fibrous scar tissue. Collectively, this creates a thickening of the intimal layer (known in some tissues as “neointimal hyperplasia”) that narrows the lumen of the passageway and causes loss of function in the tissue supplied by the particular passageway. Stenosis (or “restenosis” if the problem recurs after an initially successful attempt to open a blocked passageway) may occur during virtually any manipulation that attempts to relieve obstruction of the passageway. It may be severe enough that the passageway is reobstructed shortly after the implantation of the device.

Infection is another complication that can occur after a medical device is implanted or inserted. Typically, when a needle or catheter is inserted, the area of insertion is disinfected with an antiseptic. Occasionally, the insertion site can be inadvertently contaminated, for example, when it is palpated after the application of the antiseptic. When such devices are left in place, even for a few days, local infections often result. Exudate often seeps from the insertion site. The exudate picks up skin flora, which can diffuse back into the patient along the wetted device surface, thereby causing further infection.

In addition to the complications described herein, insertable medical devices such as sensors and needles (or catheters) may be rendered ineffective due to protein absorption on the device surface. Typically, an inserted device may be encapsulated by a protein layer that gradually thickens as the absorption process continues. The thick protein layer may interfere with the detection capability of a sensor, or the absorption of medicaments and/or nutrients that are being administered through the needle or catheter. In certain instances, the protein encapsulation process, together with risk of infection, makes it necessary to replace the needle every two to three days. Frequent replacement of the inserted devices is not only inconvenient, but also poses greater risks of introducing infectious organisms.

Applying a coating to an implantable or insertable medical device can improve the biocompatibility and efficiency of the device. For a patient, greater biocompatibility of the device means less infection and systemic reaction. Greater efficiency of the medical devices may eliminate ancillary treatments, or the necessity of replacing the devices. Hence, there remains a need in the art for coating materials that help to improve the performance of these medical devices.

The clinical performance of many medical devices (e.g., intravascular devices, such as stent grafts and aneurysm coils) depends upon the device being effectively anchored into the surrounding tissue to provide either structural support or to facilitate scarring and healing. Effective attachment of the device into the surrounding tissue, however, is not always readily achieved. One reason for ineffective attachment is that implantable medical devices generally are composed of materials that are highly biocompatible and designed to reduce the host tissue response. These materials (e.g., stainless steel, titanium based alloys, fluoropolymers, and ceramics) typically do not provide a good substrate for host tissue attachment and ingrowth during the scarring process. As a result of poor attachment between the device and the host tissue, devices can have a tendency to migrate within the vessel or tissue in which they are implanted. The extent to which a particular type of medical device can move or migrate after implantation depends on a variety of factors including the type and design of the device, the material(s) from which the device is formed, the mechanical attributes (e.g., flexibility and ability to conform to the surrounding geometry at the implantation site), the surface properties, and the porosity of the device or device surface. The tendency of a device to loosen after implantation also depends on the type of tissue and the geometry at the treatment site, where the ability of the tissue to conform around the device generally can help to secure the device in the implantation site. Device migration can result in device failure and, depending on the type and location of the device, can lead to leakage, aneurysm rupture, vessel occlusion, infarction, and/or damage to the surrounding tissue.

Numerous biological, chemical, and mechanical approaches have been proposed to secure implantable intravascular devices in place in the body.

The medical device may be anchored mechanically to biological tissue, for example, by physical or mechanical means (e.g., screws, cements, fasteners, such as sutures or staples) or by friction. Mechanical attachment of a device to the site can be effected by including in the design of the device mechanical means for fastening it into the surrounding tissue. For example, the device may include metallic spikes, anchors, hooks, barbs, pins, clamps, or a flange or lip to affix the device in place (see, e.g., U.S. Pat. Nos. 4,523,592; 6,309,416; 6,302,905; and 6,152,937). A disadvantage of mechanical fasteners, however, is that they can damage the tissue or vessel wall when the device is deployed and may not form a seal between the neck of the graft and the vessel wall. Other methods for preventing device migration have focused on mechanically altering the surface characteristics of the device. One such approach involves scoring or abrading the surface of the implant. The roughened surfaces promote cell, bone or tissue adhesion for better affixing of the implants in the body (see, e.g., WO 96/29030A1). Devices including porous surfaces have been developed to promote tissue ingrowth during the healing process which may facilitate attachment of the device to the treatment site.

Chemical or biological modifications of the device surface have been used to enhance the healing process and/or adhesion between an implantable medical device and the surrounding host tissue. In one approach, implantable medical devices have been developed which permit infiltration by specific desirable tissue cells. One type of tissue infiltration involves the process known as “endothelialization”, i.e., migration of endothelial cells from adjacent tissue onto or into the device surface. Methods for promoting endothelialization have included applying a porous coating to the device which allows tissue growth into the interstices of the implant surface (see, e.g., WO 96/37165A1). Other efforts at improving host tissue ingrowth capability and adhesion of the implant to host tissue have involved including an electrically charged or ionic material (e.g., fluoropolymer) in the tissue-contacting surface of the device (see, e.g., WO 95/19796A1; J. E. Davies, in Surface Characterization of Biomaterials, B. D. Ratner, ed., pp. 219-234 (1988); and U.S. Pat. No. 5,876,743); biocompatible organic polymers (e.g., polymers substituted with carbon, sulfur or phosphorous oxyacid groups) to promote osteogenesis at the host-implant interface (see, e.g., U.S. Pat. No. 4,795,475); and coatings made from biological materials (e.g., collagen) to enhance tissue repair, growth and adaptation at the implant-tissue interface (e.g., U.S. Pat. No. 5,002,583).

The above-described modifications, however, have failed to provide a satisfactory long-term solution to the problem of device migration. Thus, there is still a need for an effective, long-lasting and biocompatible approach for anchoring implantable intravascular devices into or onto biological tissues.

### BRIEF SUMMARY OF THE INVENTION

Briefly stated, in one embodiment, the present invention provides a medical device combined with a polymer composition, and methods of making and using the same.

One embodiment of the present invention provides a device comprising: an insertable medical device; and a polymeric coating composition comprising a bioerodable diblock copolymer of Formula: X—Y (m:n) having a molecular weight of at least 7,500, wherein,

X is a hydrophilic poly(alkylene oxide) having a molecular weight of at least 3,500,

Y is a hydrophobic polyester,

m represents a weight percentage of X based on a total weight of the diblock copolymer,

n represents a weight percentage of Y based on the total weight of the diblock copolymer, and

m+n=100.

In various embodiments, the diblock copolymer comprises MePEG and PDLLA, and m:n is about 65:35, 60:40, 50:50, 45:55, 40:60, 35;65, 30:70, 25:75, 20:80, 15:85 or 10:90.

In various embodiments, the polymeric coating composition may further comprise a therapeutic agent, including one or more anti-infective agents, anti-fibrosis agents, anticancer agents, anti-inflammatory and fibrosing agents.

Another embodiment of the present invention provides a method of preparing an insertable medical device comprising: coating the insertable medical device with a polymeric coating composition comprising a bioerodable diblock copolymer of Formula: X—Y (m:n) having a molecular weight of at least 7,500, wherein,

X is a hydrophilic poly(alkylene oxide) having a molecular weight of at least 3,500,

Y is a hydrophobic polyester,

m represents a weight percentage of X based on a total weight of the diblock copolymer,

n represents a weight percentage of Y based on the total weight of the diblock copolymer, and

m+n=100.

In various embodiments, the diblock copolymer comprises MePEG and PDLLA, and m:n is about 65:35, 60:40, 50:50, 45:55, 40:60, 35;65, 30:70, 25:75, 20:80, 15:85 or 10:90.

In various embodiments, the polymeric coating composition may further comprise a therapeutic agent, including one or more anti-infective agents, anti-fibrosis agents, anticancer agents, anti-inflammatory and fibrosing agents.

A further embodiment of the present invention provides a method of reducing surgical adhesion comprising: placing a mesh coated with a polymeric coating composition at a surgical site of a host, the polymeric coating composition comprising a bioerodable diblock copolymer of Formula: X—Y (m:n) having a molecular weight of at least 7,500, wherein,

X is a hydrophilic poly(alkylene oxide) having a molecular weight of at least 3,500,

Y is a hydrophobic polyester,

m represents a weight percentage of X based on a total weight of the diblock copolymer,

n represents a weight percentage of Y based on the total weight of the diblock copolymer, and

m+n=100.

In various embodiments, the diblock copolymer comprises MePEG and PDLLA, and m:n is about 65:35, 60:40, 50:50, 45:55, 40:60, 35;65, 30:70, 25:75, 20:80, 15:85 or 10:90.

In various embodiments, the polymeric coating composition may further comprise a therapeutic agent, including one or more anti-fibrosis agents, anticancer agents, anti-inflammatory agents

Another embodiment of the present invention provides a method of treating aneurysm comprising: delivering an injectable formulation comprising microparticles to an aneurysm sac, the microparticles being coated with a polymeric coating composition comprising a bioerodable diblock copolymer of Formula: X—Y (m:n) having a molecular weight of at least 7,500, wherein,

X is a hydrophilic poly(alkylene oxide) having a molecular weight of at least 3,500,

Y is a hydrophobic polyester,

m represents a weight percentage of X based on a total weight of the diblock copolymer,

n represents a weight percentage of Y based on the total weight of the diblock copolymer, and

m+n=100.

In various embodiments, the diblock copolymer comprises MePEG and PDLLA, and m:n is about 65:35 or 60:40.

In various embodiments, the polymeric coating composition may further comprise a therapeutic agent, including one or more fibrosing agents.

Another embodiment of the present invention provides a method of preparing an injectable formulation having microparticles comprising: mixing microparticles and a diblock copolymer in a solvent to provide a suspension, the diblock copolymer being represented by Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100; and spray-drying the suspension to provided diblock copolymer-coated microparticles.

In various embodiments, the microparticles are silk particles.

In various embodiments, the diblock copolymer comprises MePEG and PDLLA, and m:n is about 65:35 or 60:40.

In various embodiments, the polymeric coating composition may further comprise a therapeutic agent, including one or more anti-infective agents, anti-fibrosis agents, anticancer agents, anti-inflammatory and fibrosing agents.

A further embodiment of the present invention provides a method of extending the patency of an insertable medical device comprising coating the insertable medical device with a polymeric coating composition comprising a bioerodable diblock copolymer of Formula: X—Y (m:n) having a molecular weight of at least 7,500, wherein,

X is a hydrophilic poly(alkylene oxide) having a molecular weight of at least 3,500,

Y is a hydrophobic polyester,

m represents a weight percentage of X based on a total weight of the diblock copolymer,

n represents a weight percentage of Y based on the total weight of the diblock copolymer, and

m+n=100.

In various embodiments, the diblock copolymer comprises MePEG and PDLLA, and m:n is about 60:40.

In various embodiments, the polymeric coating composition may further comprise a therapeutic agent, including one or more anti-infective agents, anti-fibrosis agents, anticancer agents, anti-inflammatory agents.

### DETAILED DESCRIPTION OF THE INVENTION

Prior to setting forth the invention, it may be helpful to an understanding thereof to first set forth definitions of certain terms that are used herein.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to either one polymer or a mixture comprising two or more polymers. As used herein, the term “about” means ±15%.

In an exemplary embodiment, the present invention relates to a bioerodable polymeric coating composition that enhances the biocompatibility and efficiency of medical devices that are inserted or implanted in patients.

“Inserted” refers to a device for which at least a portion has been introduced into a host. A device such as an implant may be inserted into body tissue, for example, through the skin (percutaneously), into various types of tissue, such as muscle, bone, cartilage, tendons, fascia, and the like, or into a body lumen (e.g., a blood vessel) or cavity. A device is partially inserted when some of the device reaches, or extends to the outside of, a host. Devices may also be placed into open lumens such as urinary, nasal, rectum and oral cavities.

“Implanted” refers to an implant device that is placed completely (i.e., the whole implant resides within the host) or partially within a host. An implant or other device is partially implanted when some of the device reaches, protrudes, or extends to the outside of, a host.

“Insertable device” or “implantable device” refers to a device that may be inserted or implanted into a host.

“Host”, “person”, “subject”, “patient”, “individual” and the like are used synonymously to refer to the living being into which a device or implant of the present invention is inserted or implanted. The host may be a human or non-human animal.

As noted above, the present invention relates to an insertable or implantable device coated with a polymeric coating composition. The polymeric coating composition comprises a bioerodable diblock copolymer, and optionally a therapeutic agent. The polymeric coating composition may further comprise an additional polymer, which may be bioerodable or non-bioerodable. A material is bioerodable (or biodegradable) if it safely degrades into non-toxic substances or otherwise erodes away in living tissue/fluid. The process can be fairly rapid as with water-soluble materials (e.g., low molecular weight PEG), or can take place over a more extended time period when the process depends on a hydrolysis reaction(s), e.g., as would be the case with polyesters based on hydroxy acid residues.

It is further desirable that the coating material is biocompatible. The term “biocompatible” means that the coating material does not induce an adverse response when exposed to living tissue. An adverse response can be an infection, an immune response elicited by the device as a “foreign body”, protein encapsulation of the device, or any other processes that reduce the effectiveness of the medical device. As will be discussed in more detail herein, the biocompatibility of the coating material can be enhanced by one or more therapeutic agents incorporated in the coating material.

The present invention provides a medical device comprising a polymeric coating composition. The polymeric coating composition possesses unique and tunable physical characteristics, which make it suitable for coating medical devices of diverse configurations and functions. Medical devices that may be coated with the polymeric coating composition include, but are not limited to, meshes, needles, catheters, implantable sensors, and injectable microparticles. In addition, the present invention provides methods for making the medical device and methods for extending patency of the medical device. Furthermore, the present invention provides methods of using the medical device, such as using coated meshes in treating or reducing surgical adhesion and using coated injectable formulations that comprise microparticles in treating aneurysm.

A. Medical Devices Combined With Polymeric Coating Compositions and Methods for Preparing the Same

In one aspect, the present invention provides a medical device that comprises a polymeric coating composition. The polymeric coating composition comprises a bioerodable diblock copolymer, and may further comprise a second polymer, one or more therapeutic agents, a buffer, and a solvent. The medical devices that may be coated with the polymeric coating composition include various types of insertable medical devices, such as needles, catheters, meshes, and injectable microparticles.

In a related aspect, the present invention provides a method for preparing a medical device that comprises a polymeric coating composition. The resulting coated medical devices typically have an extended patency due to the polymeric coating.

1) Polymeric Coating Compositions

In certain embodiments, it is described herein a medical device combined with a polymeric coating composition, the polymer composition comprising a bioerodable diblock copolymer of Formula: X—Y (m:n) having a molecular weight of at least 7,500, wherein X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In various embodiments, the diblock copolymer is present in the polymeric coating composition in about 2%, 4%, 10%, 15%, 20%, 30%, 40%, 50%, 60%, 70%, 80%, 90% or 100%. Other components of the polymeric coating composition may include one or more therapeutic agents, one or more additional polymers, and a solvent or buffer.

As will be discussed in detail herein, the polymeric coating composition of the present invention can be combined with a variety of insertable or implantable medical devices. Advantageously, the polymeric coating composition can be tailor to accommodate diverse structures of many medical devices. For example, the polymeric coating composition can be fully or partially coated on certain medical devices, or incorporated into divets, channels, and voids of other medical devices.

In certain embodiments, the polymeric coating composition further comprises a therapeutic agent, which enhances the biocompatibility of the medical device. Suitable therapeutic agents include, but are not limited to anti-fibrosis agents, anti-infective agents, anti-proliferative agents, fibrosis inducing agents, and a combination thereof. The polymeric coating composition is particularly suited for incorporating a hydrophobic therapeutic agent.

In other embodiments, the polymeric coating composition further comprises an additional polymer. The additional polymer mixes with the diblock copolymer to formulate coating compositions having a wider range of physio-chemical properties such as viscoelasticity, mechanical strength, hydrophilicity, rate of erosion, and the like.

In other embodiments, the polymeric coating composition further comprises one or more other components such as a buffer, solvent, colorant, and surfactant.

(A) Diblock Copolymer

“Diblock” copolymer refers to a linear chain macromolecule comprising two subchains (or blocks) covalently joined to each other. One block comprises residues of a first type of monomer(s) and the other block of a second type of monomer(s).

A diblock copolymer typically exhibits the combined physio-chemical properties, such as hydrophilicity, elasticity, swellability and biocompatibility, of the two blocks. As will be discussed further in detail herein, the physio-chemical properties of the diblock copolymer can therefore be modulated by adjusting the relative amount of each block.

According to the present invention, the diblock copolymer comprises a hydrophilic block X and a hydrophobic block Y. More specifically, the X block is a polyether comprising alkylene oxide residues. The Y block is a polyester comprising hydroxy acid residues.

Alkylene oxide represents the minimal repeating unit of poly(alkylene oxide). “Alkylene oxide residue” refers to a diradical of formula —Rₓ—O—, wherein Rₓ is an alkyl group having 1-6 carbons in a linear or branched arrangement. Examples of alkylene oxide residues include, but are not limited to: ethylene oxide, propylene oxide and 1-methylethyl oxide.

In certain embodiments, the X block is a homopolymer comprising residues of the same alkylene oxide. For example, the X block can be a homopolymer comprising ethylene oxide residues, also referred to as polyethylene glycol, i.e., PEG.

Preferably, the X block is terminated with an alkyl moiety. Examples of the terminating alkyl moiety include methyl, ethyl and propyl. In one embodiment, the X block is a methyl-terminated polyethylene glycol, also referred to as MePEG.

In other embodiments, the X block comprises residues of more than one type of alkylene oxide. For example, the X block may comprise residues of ethylene oxide and propylene oxide. Accordingly, the X block itself is a copolymer. For instance, the X block may be poly(ethylene oxide)-co-poly(propylene oxide), e.g., PLURONIC® and PLURONIC R® series of polymers (BASF Corporation, Mount Olive, N.J.)

Typically, poly(alkylene oxide) is hydrophilic. In particular, PEGs (including MePEG) are water soluble in a broad molecular weight range. The hydrophilicity of the X block contributes to its physio-chemical properties in a tissue environment, which contains mainly aqueous fluid. In particular, the X block is bioerodable.

The molecular weight of the X block further contributes to its physio-chemical properties, such as solubility, viscoelasticity and rate of bioerosion. Typically, higher molecular weight leads to lower water solubility and slower rate of erosion. The X block, i.e., poly(alkylene oxide), of the present invention has a molecular weight of at least 3,500. In one embodiment, the X block has a molecular weight of at least about 5,000. In other embodiments, the X block has a molecular weight of at least 6,500, at least 8,000, or at least 10,000.

“Hydroxy acid” refers to a hydroxy-substituted carboxylic acid or a cyclic ester. A hydroxy acid is essentially a monomer having two functionalities: a hydroxy group and a carboxylic acid. The bifunctional nature of the hydroxy acid makes it a suitable starting material for forming the polyester block (Y) of the diblock copolymer. According to the present invention, a hydroxy acid can also be in the form of a cyclic ester, which is a reactive equivalent of a hydroxy-substituted carboxylic acid. Examples of the suitable hydroxy acids include, but are not limited to: lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one and 1,5-dioxepan-2-one.

Polyesters based on the residues of the above hydroxy acids are well known for their bioerodibility. They typically disintegrate in the living tissue through hydrolytic degradation of the ester bonds. The degradation time is a function of several factors, including the chemical composition, molecular weight and crystallinity of the polyester.

In certain embodiments, the Y block is poly(D,L-lactide), also referred to as PDLLA. In other embodiments, the Y block is poly(glycolide), i.e., PGA. In other embodiments, the Y block is a copolymer of poly(lactide-co-glycolide), i.e., PLGA.

The X block is covalently joined with the Y block by an ester bond. More specifically, when using poly(alkylene oxide) as a starting material, a hydroxy terminus of the poly(alkylene oxide) induces the polymerization of a hydroxy acid monomer by reacting with the carboxylic acid functionality. The chain extension is achieved when the hydroxy functionality of the hydroxy acid continues to react with the carboxylic acid functionality of another molecule of hydroxy acid.

As illustrated in Scheme I, the polymerization of the polyester block is initiated by a ring-opening reaction in which the hydroxy terminus of MePEG reacts with a D,L-lactide, which is a cyclic ester of a dimeric lactic acid. The polymerization can be achieved with or without a catalyst.

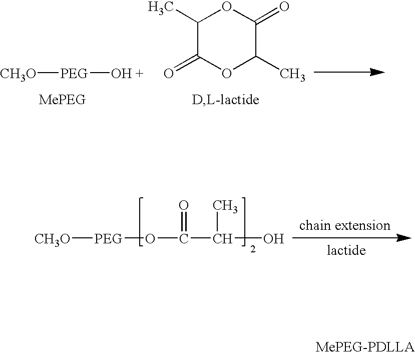

When prepared according to Scheme I, the molecular weight of the diblock copolymer can be controlled by selecting a specific molecular weight of the X block as a starting material, and by selecting a specific weight ratio of the X block and the hydroxy acid (e.g., lactide) monomer. Assuming all of the hydroxy acid monomers are consumed, the weight ratio of the X block to the hydroxy acid monomer is equivalent to the weight ratio of the X block to the Y block formed by the polymerization of the hydroxy acid monomers.

As noted above, the diblock copolymer can be represented by Formula X—Y (m:n), wherein m and n are the respective weight percentages of the X block and Y block, and m+n=100. Thus, the molecular weight of the diblock copolymer can be calculated by:

$$molecular weight of the X block + \frac{n}{m} \times molecular weight of the X block$$

Thus, the molecular weight of the diblock copolymer is proportional to the molecular weight of the X block, and is further determined by the weight ratio of the Y block over the X block (i.e., n/m).

In accordance to the present invention, the molecular weight of the diblock copolymer is at least 7,500. In various embodiments, the molecular weight of the diblock copolymer is at least 8,500, at least 10,000, at least 15,000, at least 50,000, at least 75,000, and at least 100,000.

In various embodiments, m:n is about 65:35, 60:40, 50:50, 40:60, 30:70, 20:80 or 10:90. Table 1 presents the molecular weights (MW) of an exemplary diblock copolymer (MePEG-PDLLA) at various weight ratios. Table 1 shows a clear trend that a higher ratio the hydrophobic block leads to a higher overall molecular weight of the diblock copolymer.

TABLE 1

| | | MW of MePEG-PDLLA |
|---|---|---|
| m (%) | n (%) | MePEG MW = 5000 |
| 65 | 35 | 7,700 |
| 60 | 40 | 8,333 |
| 50 | 50 | 10,000 |
| 40 | 60 | 12,500 |
| 30 | 70 | 16,667 |
| 20 | 80 | 25,000 |
| 10 | 90 | 50,000 |

The diblock copolymer of the present invention displays unique physio-chemical properties based on the combined properties of its constituent blocks. In particular, the physio-chemical properties of the diblock copolymers can be tuned by controlling the relative weight ratio of the X block to Y block. This enables diverse coating compositions suitable for a range of medical devices.

The diblock copolymer is insoluble in water due to its hydrophobic content. However, the diblock copolymer can swell in water. In addition, the hydrophilic content improves the wettability to the diblock copolymer. As a result, the polymeric coating composition imparts lubrication and biocompatibility to the coated medical device.

The diblock copolymer further exhibits favorable viscoelastic properties, which promote a conformal and durable coating for a variety of medical devices. The diblock copolymer is particularly suitable for coating devices with irregular surface topography and certain degrees of porosity, such as a mesh, as defined herein.

Moreover, the diblock copolymer is bioerodable. The rate of erosion depends on factors such as the chemical nature of the blocks, the weight ratio of the X block to Y block, and the overall molecular weight. In various embodiments, the diblock copolymer may erode in about 3 days, a week, two weeks, one month, two months, 6 months, or 12 months. Typically, a higher content of the hydrophobic block tends to take longer to erode. For example, MePEG-PDLLA (65:35), wherein MePEG has a molecular weight of about 5000, erodes completely in about 4 days in vitro in phosphate buffer saline. In comparison, it typically takes at least 3-6 months or even longer in some cases (e.g., about 12-18 months) for PDLLA to completely erode.

In certain embodiments, erosion of the polymeric coating composition enhances the biocompatibility of a coated medical device. For example, protein absorption on the surface of the medical device can be disrupted when the coating composition erodes. The process is also referred to as “slough off”. Effective slough off re-exposes the medical device, such as a surface of a sensor, which has been previously encapsulated by a protein layer. Slough off may also re-canalize an opening of a needle or catheter that has been previously blocked.

The diblock copolymer is further compatible with incorporating one or more therapeutic agents. The hydrophobic block provides particular advantages for accommodating therapeutic agents that are substantially hydrophobic. Advantageously, the polymeric coating composition allows for a controlled or sustained release of the therapeutic agent from the coating. “Release of an agent” can be measured as a statistically significant presence of the therapeutic agent, or a subcomponent thereof, which has disassociated from the medical device.

Depending on the weight ratio of the X to Y blocks, as well as the molecular weight of the X block, the diblock copolymer typically has a glass transition temperature (Tg) ranges from −5° C. to 60° C. More typically, the diblock copolymer has a Tg ranging from 5° C. to 20° C. In certain embodiments, the diblock copolymer is a gel at room temperature. In other embodiments, the diblock copolymer is a viscous liquid, semi-solid at room temperature. In yet other embodiments, the diblock copolymer is solid at room temperature.

(B) Therapeutic Agents

In addition to the diblock copolymer, the polymeric coating composition of the present invention may further comprise a therapeutic agent.

“Therapeutic agent,” “bioactive agent” and “drug” are used interchangeably herein to refer to a chemical material or compound suitable for administration to a patient and that induces a desired effect. The terms include agents that are therapeutically effective as well as prophylactically effective. Also included are derivatives and analogs of those compounds or classes of compounds specifically mentioned that also induce the desired effect.

In certain embodiments, the therapeutic agent incorporated in the polymeric coating composition enhances the biocompatibility and efficiency of the coated medical device. For example, a therapeutic agent that inhibits infection can prevent or reduce local infection at or near the site of the implantation. Therapeutic agents that inhibit fibrosis or cell proliferation can prevent the formation of fibrotic tissue, or protein absorption on the device. When applied to a percutaneously insertable surface of an insertable or implantable medical device, polymeric coating compositions incorporating these therapeutic agents can substantially extend the patency of the device.

Under certain circumstances, fibrotic tissue formation at the implantation site is beneficial to an implanted medical device. For example, adhesion or fibrosis in the tissue surrounding the medical device can facilitate “anchoring” of the implanted device in situ, thus enhancing the efficacy of the device. Accordingly, in other embodiments, the polymeric coating composition of the present invention may comprise a therapeutic agent that induces fibrosis.

In certain embodiments, the polymeric coating material comprises about 0.1% to 50%, from about 0.5% to 30%, or from about 3% to 20% of one or more therapeutic agents.

Suitable therapeutic agents of the present invention therefore include, but are not limited to, anti-infective agents, anti-fibrosis agents, anticancer agents, anti-inflammatory and fibrosing agents.

a) Anti-Infective Agents

An “anti-infective agent” refers to a chemical entity or a composition of chemical entities that prevent infections near or at the site of the agent. Infections are characterized by the accumulation and proliferation of microorganisms, such as bacteria, viruses, fungi, and the like. The anti-infective agent is expected to inhibit these processes at a statistically significant level at or near the site of the agent.

Representative examples of the anti-infective agents include a quaternary compound, a phenolic compound, an iodinated compound, a silver compound or an acidic-anionic compound. Examples of anti-infective agents include one or more of 2-bromo-2-nitropropane-1,3-diol (e.g., BRONOPOL), Irgasan (TRICLOSAN), polyhexanide (also known as polyhexamethylene biguanide) (e.g., VANTOCIL IB, COSMOCIL CQ, or BAQUACIL), benzalkonium chloride, benzethonium chloride, cetylpyradinium chloride, stearalkonium chloride, phenol, cresol, aminophenol, iodine, iodide, 8-hydroxyquinolone, and chlorhexidine.

Other bioactive agents, which have been shown to have anti-infective characteristics, in addition to other therapeutic uses, may be used in the present compositions. For example, the anti-infective agent may be a chemotherapeutic agent. Numerous chemotherapeutic agents have been identified, which have potent antimicrobial activity at extremely low doses. Examples of these agents are described in U.S. Published Patent Application No. 20040043052, which is incorporated herein in its entirety, and include anthracyclines (e.g., doxorubicin and mitoxantrone), fluoropyrimidines (e.g., 5-fluorouracil (5-FU)), folic acid antagonists (e.g., methotrexate), podophylotoxins (e.g., etoposide), camptothecins, hydroxyureas, and platinum complexes (e.g., capsulation), and analogs or derivatives thereof.

Exemplary anthracyclines include doxorubicin, daunorubicin, idarubicin, epirubicin, pirarubicin, zorubicin, carubicin, anthramycin, mitoxantrone, menogaril, nogalamycin, aclacinomycin A, olivomycin A, chromomycin A₃, plicamycin, FCE 23762, a doxorubicin derivative, annamycin, ruboxyl, anthracycline disaccharide doxorubicin analog, 2-pyrrolinodoxorubicin, disaccharide doxorubicin analogs, 4-demethoxy-7-O-[2,6-dideoxy-4-O-(2,3,6-trideoxy-3-amino-α-L-lyxo-hexopyranosyl)-α-L-lyxo-hexopyranosyl]adriamicinone doxorubicin disaccharide analog, 2-pyrrolinodoxorubicin, morpholinyl doxorubicin analogs, enaminomalonyl-β-alanine doxorubicin derivatives, cephalosporin doxorubicin derivatives, hydroxyrubicin, methoxymorpholino doxorubicin derivative, (6-maleimidocaproyl)hydrazone doxorubicin derivative, N-(5,5-diacetoxypent-1-yl) doxorubicin, FCE 23762 methoxymorpholinyl doxorubicin derivative, N-hydroxysuccinimide ester doxorubicin derivatives, polydeoxynucleotide doxorubicin derivatives, morpholinyl doxorubicin derivatives, mitoxantrone doxorubicin analog, AD198 doxorubicin analog, 4-demethoxy-3′-N-trifluoroacetyldoxorubicin, 4′-epidoxorubicin, alkylating cyanomorpholino doxorubicin derivative, deoxydihydroiodooxorubicin, adriblastin, 4′-deoxydoxorubicin, 4-demethyoxy-4′-o-methyldoxorubicin, 3′-deamino-3′-hydroxydoxorubicin, 4-demethyoxy doxorubicin analogs, N-L-leucyl doxorubicin derivatives, 3′-deamino-3′-(4-methoxy-1-piperidinyl) doxorubicin derivatives, 3′-deamino-3′-(4-mortholinyl) doxorubicin derivatives, 4′-deoxydoxorubicin and 4′-o-methyldoxorubicin, aglycone doxorubicin derivatives, SM 5887, MX-2,4′-deoxy-13(S)-dihydro-4′-iododoxorubicin, morpholinyl doxorubicin derivatives, 3′-deamino-3′-(4-methoxy-1-piperidinyl) doxorubicin derivatives, doxorubicin-14-valerate, morpholinodoxorubicin, 3′-deamino-3′-(3″-cyano-4″-morpholinyl doxorubicin, 3′-deamino-3′-(3″-cyano-4″-morpholinyl)-13-dihydoxorubicin, (3′-deamino-3′-(3″-cyano-4″-morpholinyl) daunorubicin, 3′-deamino-3′-(3″-cyano-4″-morpholinyl)-3-dihydrodaunorubicin, 3′-deamino-3′-(4″-morpholinyl-5-iminodoxorubicin, 3′-deamino-3′-(4-methoxy-1-piperidinyl) doxorubicin derivatives, and 3-deamino-3-(4-morpholinyl) doxorubicin derivatives.

Exemplary fluoropyrimidine analogs include 5-fluorouracil, or an analog or derivative thereof, including carmofur, doxifluridine, emitefur, tegafur, and floxuridine. Other exemplary fluoropyrimidine analogs include 5-FudR (5-fluoro-deoxyuridine), or an analog or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Other representative examples of fluoropyrimidine analogs include N3-alkylated analogs of 5-fluorouracil, 5-fluorouracil derivatives with 1,4-oxaheteroepane moieties, 5-fluorouracil and nucleoside analogs, cis- and trans-5-fluoro-5,6-dihydro-6-alkoxyuracil, cyclopentane 5-fluorouracil analogs, A-OT-fluorouracil, N4-trimethoxybenzoyl-5′-deoxy-5-fluorocytidine and 5′-deoxy-5-fluorouridine, 1-hexylcarbamoyl-5-fluorouracil, B-3839, uracil-1-(2-tetrahydrofuryl)-5-fluorouracil, 1-(2′-deoxy-2′-fluoro-β-D-arabinofuranosyl)-5-fluorouracil, doxifluridine, 5′-deoxy-5-fluorouridine, 1-acetyl-3-O-toluyl-5-fluorouracil, 5-fluorouracil-m-formylbenzene-sulfonate, N′-(2-furanidyl)-5-fluorouracil, and 1-(2-tetrahydrofuryl)-5-fluorouracil.

Exemplary folic acid antagonists include methotrexate or derivatives or analogs thereof, such as edatrexate, trimetrexate, raltitrexed, piritrexim, denopterin, yomudex, pteropterin. Other representative examples include 6-S-aminoacyloxymethyl mercaptopurine derivatives, 6-mercaptopurine (6-MP), 7,8-polymethyleneimidazo-1,3,2-diazaphosphorines, azathioprine, methyl-D-glucopyranoside mercaptopurine derivatives and s-alkynyl mercaptopurine derivatives, indoline ring and a modified ornithine or glutamic acid-bearing methotrexate derivatives, alkyl-substituted benzene ring C bearing methotrexate derivatives, benzoxazine or benzothiazine moiety-bearing methotrexate derivatives, 10-deazaminopterin analogs, 5-deazaminopterin and 5,10-dideazaminopterin methotrexate analogs, indoline moiety-bearing methotrexate derivatives, lipophilic amide methotrexate derivatives, L-threo-(2S,4S)-4-fluoroglutamic acid and DL-3,3-difluoroglutamic acid-containing methotrexate analogs, methotrexate tetrahydroquinazoline analog, N-(α-aminoacyl) methotrexate derivatives, biotin methotrexate derivatives, D-glutamic acid or D-erythrou, threo-4-fluoroglutamic acid methotrexate analogs, β,γ-methano methotrexate analogs, 10-deazaminopterin (10-EDAM) analog, γ-tetrazole methotrexate analog, N-(L-α-aminoacyl) methotrexate derivatives, meta and ortho isomers of aminopterin, hydroxymethylmethotrexate, γ-fluoromethotrexate, polyglutamyl methotrexate derivatives, gem-diphosphonate methotrexate analogs, α- and γ-substituted methotrexate analogs, 5-methyl-5-deaza methotrexate analogs, Nδ-acyl-Nα-(4-amino-4-deoxypteroyl)-L-ornithine derivatives, 8-deaza methotrexate analogs, acivicin methotrexate analog, polymeric platinol methotrexate derivative, methotrexate-γ-dimyristoylphophatidylethanolamine, methotrexate polyglutamate analogs, poly-γ-glutamyl methotrexate derivatives, deoxyuridylate methotrexate derivatives, iodoacetyl lysine methotrexate analog, 2-omega-diaminoalkanoid acid-containing methotrexate analogs, polyglutamate methotrexate derivatives, 5-methyl-5-deaza analogs, quinazoline methotrexate analog, pyrazine methotrexate analog, cysteic acid and homocysteic acid methotrexate analogs, γ-tert-butyl methotrexate esters, fluorinated methotrexate analogs, folate methotrexate analog, phosphonoglutamic acid analogs, poly (L-lysine) methotrexate conjugates, dilysine and trilysine methotrexate derivates, 7-hydroxymethotrexate, poly-γ-glutamyl methotrexate analogs, 3′,5′-dichloromethotrexate, diazoketone and chloromethylketone methotrexate analogs, 10-propargylaminopterin and alkyl methotrexate homologs, lectin derivatives of methotrexate, polyglutamate methotrexate derivatives, halogentated methotrexate derivatives, 8-alkyl-7,8-dihydro analogs, 7-methyl methotrexate derivatives and dichloromethotrexate, lipophilic methotrexate derivatives and 3′,5′-dichloromethotrexate, deaza amethopterin analogs, MX068, and cysteic acid and homocysteic acid methotrexate analogs.

Exemplary podophyllotoxins include etoposide, teniposide, Cu(II)-VP-16 (etoposide) complex, pyrrolecarboxamidino-bearing etoposide analogs, 4β-amino etoposide analogs, γ-lactone ring-modified arylamino etoposide analogs, N-glucosyl etoposide analog, etoposide A-ring analogs, 4′-deshydroxy-4′-methyl etoposide, pendulum ring etoposide analogs, and E-ring desoxy etoposie analogs.

Exemplary camptothecins include topotecan, irinotecan (CPT-11), 9-aminocamptothecin, 21-lactam-20(S)-camptothecin, 10,11-methylenedioxy-camptothecin, SN-38, 9-nitrocamptothecin, and 10-hydroxycamptothecin.

Exemplary platinum complexes include complexes of Pt(II) or Pt(IV), cisplatin, carboplatin, oxaliplatin, and miboplatin. Other representative examples of platinum compounds include (CPA)₂Pt[DOLYM] and (DACH)Pt[DOLYM] cisplatin, Cis-[PtCl₂(4,7-H-5-methyl-7-oxo] 1,2,4[triazolo[1,5-a]pyrimidine)₂], [Pt(cis-1,4-DACH)(trans-Cl₂)(CBDCA)].½MeOH cisplatin, 4-pyridoxate diammine hydroxy platinum, Pt(II) . . . Pt(II) (Pt₂[NHCHN(C(CH₂)(CH₃))]₄), 254-S cisplatin analog, o-phenylenediamine ligand bearing cisplatin analogs, trans, cis-[Pt(OAc)₂I₂(en)], estrogenic 1,2-diarylethylenediamine ligand (with sulfur-containing amino acids and glutathione) bearing cisplatin analogs, cis-1,4-diaminocyclohexane cisplatin analogs, 5′ orientational isomer of cis-[Pt(NH₃)(4-aminoTEMP-O){d(GpG)}], chelating diamine-bearing cisplatin analogs, 1,2-diarylethyleneamine ligand-bearing cisplatin analogs, (ethylenediamine)platinum(II) complexes, CI-973 cisplatin analog, cis-diaminedichloroplatinum(II) and its analogs cis-1,1-cyclobutanedicarbosylato(2R)-2-methyl-1,4-butanediamineplatinum(II) and cis-diammine(glycolato)platinum, cis-amine-cyclohexylamine-dichloroplatinum(II), gem-diphosphonate cisplatin analogs, (meso-1,2-bis(2,6-dichloro-4-hydroxyplenyl)ethylenediamine) dichloroplatinum(II), cisplatin analogs containing a tethered dansyl group, platinum(II) polyamines, cis-(3H)dichloro(ethylenediamine)platinum(II), trans-diamminedichloroplatinum(II) and cis-(Pt(NH₃)₂(N₃-cytosine)CI), 3H-cis-1,2-diaminocyclohexanedichloroplatinum(II) and 3H-cis-1,2-diaminocyclohexanemalonatoplatinum (II), diaminocarboxylatoplatinum, trans-(D,1)-1,2-diaminocyclohexane carrier ligand-bearing platinum analogs, aminoalkylaminoanthraquinone-derived cisplatin analogs, spiroplatin, carboplatin, iproplatin and JM40 platinum analogs, bidentate tertiary diamine-containing cisplatinum derivatives, platinum(II), platinum(IV), cis-diammine(1,1-cyclobutanedicarboxylato-) platinum(II) (carboplatin, JM8) and ethylenediammine-malonatoplatinum(II) (JM40), JM8 and JM9 cisplatin analogs, (NPr₄)₂((PtCl₄).cis-(PtCl₂-(NHMe)₂)), aliphatic tricarboxylic acid platinum complexes, and cis-dichloro(amino acid)(tert-butylamine)platinum(II) complexes.

In one embodiment, the anti-infective agent may be benzalkonium heparinate or sodium heparin. In another embodiment of the invention, the coating composition does not contain any ethylenediamine tetraacetic acid (EDTA).

The anti-infective agent can be present in the polymeric coating composition from about 0.1% to 50%, or from about 0.5% to 30%, 3% to 27%, 3%, 6%, 11%, 13%, 17%, 20%, 25% or 27% by weight.

b) Anti-Fibrotic Agents

Therapeutic agents which inhibit fibrosis or scarring are referred to herein as “anti-fibrotic agents,” “fibrosis-inhibiting agents,” “anti-scarring agents,” and the like. “Fibrosis,” “scarring,” or “fibrotic response” refers to the formation of fibrous tissue in response to injury or medical intervention. “Inhibit fibrosis,” “reduce fibrosis,” and the like are used synonymously to refer to the action of agents or compositions which result in a statistically significant decrease in the formation of fibrous tissue that can be expected to occur in the absence of the agent or composition.

The anti-fibrotic agents inhibit fibrosis through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling. In addition, numerous therapeutic agents described in this invention will have the additional benefit of also reducing tissue regeneration (the replacement of injured cells by cells of the same type) when appropriate.

The presence of the anti-fibrotic agents in the polymeric coating composition prevents scar tissue formation and/or protein encapsulation on or near the coatede medical device.

A number of anti-fibrotic agents are described, e.g., in U.S. Patent Application, “Medical Implants and Anti-Scarring Agents,” filed Nov. 10, 2004 (U.S. Ser. No. 10/986,231); and “Anti-Scarring Agents, Therapeutic Compositions, and Use Thereof,” filed May 10, 2005 (U.S. Ser. No. 60/679, 293), which applications are incorporated herein by reference in their entireties. Exemplary anti-fibrotic agents include, but are not limited to, cell cycle inhibitors (e.g., doxorubicin, mitoxantrone, TAXOTERE, vinblastine, tubercidin, paclitaxel, and analogues and derivatives thereof, podophyllotoxins (e.g., etoposide), immunomodulators (e.g., sirolimus and everolimus), heat shock protein 90 antagonists (e.g., geldanamycin) and analogues and derivatives thereof, HMGCoA reductase inhibitors (e.g., simvastatin) and analogues and derivatives thereof, inosine monophosphate dehydrogenase inhibitors (e.g., mycophenolic acid, 1-alpha-25 dihydroxy vitamin D₃) and analogues and derivatives thereof, NF kappa B inhibitors (e.g., Bay 11-7082) and analogues and derivatives thereof, antimycotic agents (e.g., sulconizole) and analogues and derivatives thereof, p38 MAP kinase inhibitors (e.g., SB202190) and analogues and derivatives thereof, and anti-angiogenic agents (e.g., halofuginone bromide) and analogues and derivatives. Additional exemplary anti-fibrotic agents include, but are not limited to, ZD-6474 (an angiogenesis inhibitor), AP-23573 (an mTOR inhibitor), synthadotin (a tubulin antagonist), S-0885 (a collagenase inhibitor), aplidine (an elongation factor-1 alpha inhibitor), ixabepilone (an epithilone), IDN-5390 (an angiogenesis inhibitor and an FGF inhibitor), SB-2723005 (an angiogenesis inhibitor), ABT-518 (an angiogenesis inhibitor), combretastatin (an angiogenesis inhibitor), anecortave acetate (an angiogenesis inhibitor), SB-715992 (a kinesin antagonist), temsirolimus (an mTOR inhibitor), adalimumab (a TNFα antagonist), erucylphosphocholine (an ATK inhibitor), alphastatin (an angiogenesis inhibitor), BXT-51072 (an NF Kappa B inhibitor), etanercept (a TNFα antagonist and TACE inhibitor), humicade (a TNFα inhibitor), and gefitinib (a tyrosine kinase inhibitor), as well as analogues and derivatives of the aforementioned.

c) Anticancer Agents

Anticancer or anti-proliferative agents suitable to be incorporated into the polymeric coating composition may act by a number of mechanisms. These agents may be antimetabolites, anti-microtubule agents, chelating agents, antibiotics or antiangiogenic agents. Exemplary anticancer agents useful in the present invention include, but are not limited to, alkylating agents such as bis(chloroethyl)amines (including cyclophosphamide, mechlorethamine, chlorambucil, and melphalan), nitrosoureas (including carmustine, estramustine, lomustine, and semustine), aziridines (including thiotepa and triethylenemelamine), alkylsulfonates including busulfan, other agents with possible alkylating agent activity (including procarbazine, cisplatin, carboplatin, dacarbazine, and hexamethylmelamine); antimetabolites such as methotrexate, mercaptopurine, thioguanine, 5-fluorouracil, cytarabine, and azacitidine; plant alkaloids such as vinca alkaloids (including vincristine, vinorelbine, and vinblastine), bleomycin, dactinomycin, anthracyclines (including daunorubicin or doxorubicin, idarubicin, epirubicin, pirarubicin, zorubicin carubicin, anthramycin, mitoxantrone, menogaril, nogalamycin, aclacinomycin A, olivomycin A, chromomycin A₃, and plicamycin), etoposide, teniposide, mithramycin, mitomycin; hormonal agents such as androgens (including testosterone, or fluoxymestrone), antiandrogens including flutamide, estrogens (including diethylstilbesterol, estradiol, ethylestradiol, and estrogen), antiestrogens including tamoxifen, progestins (including hydroxyprogesterone, progesterone, medroxyprogesterone, and megestrol acetate), adrenocorticosteroids (including hydrocortisone and prednisone), gonadotropin-releasing hormones and agonists thereof including leuprolide; aminoglutethemide (sold under the tradename CYTADREN), amscarine, asparaginase, hydroxyurea, mitotane, and quniacrine; and anti-microtubule agents including paclitaxel and docetaxol. Also included are analogues and derivatives of the aforementioned compounds. Other anticancer agents include antiangiogenic agents such as active taxanes as described above, including paclitaxel and docetaxol; angiostatic steroids including squaline; cartilage derived proteins and factors; thrombospondin; matrix metalloproteinases (including collagenases, gelatinases A and B, stromelysins 1, 2 and 3, martilysin, metalloelastase, MT1-MMP (a progelatenase), MT2-MMP, MT3-MMP, MT4-MMP, Bay 12-9566 (Bayer), AG-3340 (Agouron), CGS27023! (Novartis), and Chiroscience compounds D5140, D1927, D2163); and phytocemicals (including genistein, daidzein, leuteolin, apigenin, 3 hydroxyflavone, 2′,3′-dihydroxyflavone, 3′,4′-dihydroxyflavone, and fisetin). Anti-angiogentic agents also include active analogues and derivatives of the aforementioned antiangiogenic agents. Certain anticancer agents are also classified as antifibrotic agents. These include mitomycin C, 5-fluorouracil, interferons, D-penicillamine and β-aminoproprionitrile. Additional anticancer agents include other compounds that exhibit therapeutic activity against cancer as defined using standard tests known in the art, including in vitro cell studies, in vivo and ex vivo animal studies, and clinical human studies. Suitable tests are described in texts such as “Anticancer Drug Development Guide” (B. A. Teicher ed., Humana Press, 1997 Totowa, N.J.).

In one embodiment, the anti-microtubule agent is paclitaxel, a compound that disrupts mitosis (M-phase) by binding to tubulin to form abnormal mitotic spindles, or an analogue or derivative thereof.

The utility of the anti-microtubule agent paclitaxel, as a component of the compositions that comprise part of this invention, is demonstrated by data from a series of in vitro and in vivo experiments. Paclitaxel inhibits neutrophil activation (Jackson et al., Immunol. 90:502-10,1997), decreases T-cell response to stimuli, and inhibits T-cell function (Cao et al., J. Neuroimmunol. 108:103-11, 2000), prevents the proliferation of and induces apoptosis in synoviocytes (Hui et al., Arth. Rheum. 40:1073-84,1997), inhibits AP-1 transcription activity via reduced AP-1 binding to DNA (Hui et al., Arth. Rheum. 41:869-76, 1998), inhibits collagen induced arthritis in an animal model (Brahn et al., Arth. Rheum. 37:839-45, 1994; Oliver et al., Cellular Imunol. 157:291-9, 1994), but is non-toxic to non-proliferating cells, such as normal chondrocytes and non-proliferating synoviocytes (Hui et al., Arth. Rheum. 40:1073-84,1997).

Paclitaxel, formulations, prodrugs, epimers, isomers, analogues and derivatives thereof may be readily prepared utilizing techniques known to those skilled in the art (see, e.g., Schiff et al., Nature 277:665-667, 1979; Long and Fairchild, Cancer Research 54:4355-4361,1994; Ringel and Horwitz, J. Nat'l Cancer Inst. 83(4):288-291,1991; Pazdur et al., Cancer Treat. Rev. 19(4):351-386,1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237,1992; J. Med. Chem. 34:992-998,1991; J. Natural Prod. 57(10):1404-1410, 1994; J. Natural Prod. 57(11):1580-1583, 1994; J. Am. Chem. Soc. 110:6558-6560,1988), or obtained from a variety of commercial sources, including for example, Sigma Chemical Co., St. Louis, Mo. (T7402-from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol, phosphonoxy and carbonate derivatives of taxol, taxol 2′,7-di(sodium 1,2-benzenedicarboxylate, 10-desacetoxy-11,12-dihydrotaxol-10,12(18)-diene derivatives, prodrugs including 2′-and/or 7-O-ester, amide, thioester derivatives, (2′-and/or 7-O-carbonate derivatives), fluoro taxols, 9-deoxotaxol, 7-deoxy-9-deoxotaxol, 10-desacetoxy-7-deoxy-9-deoxotaxol, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′-succinyltaxol; 2′-(beta-alanyl)-taxol); 2′-γ-aminobutyryltaxol formate; ethylene glycol derivatives of 2′-succinyltaxol; prodrugs or derivatives having amino acids attached at either or both of the 2′ and 7 positions (R₉ and R₃, respectively); 2′-glutaryltaxol; 2′-(N,N-dimethylglycyl) taxol; 2′-(2-(N,N-dimethylamino)propionyl)taxol; 2′-orthocarboxybenzoyl taxol; 2′-aliphatic carboxylic acid derivatives of taxol, prod rugs {2′-(N,N-diethylaminopropionyl)taxol, 2′(N,N-dimethylglycyl)taxol, 7(N,N-dimethylglycyl) taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′, 7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, TAXOL (Bristol-Myers Squibb Company, New York, N.Y.) analogues with modified phenylisoserine side chains, taxotere, (N-debenzoyl-N-tert-(butoxycaronyl)-10-deacetyltaxol, cephalomannine, Taxol C, Taxol D, Taxol E, Taxol F, brevifoliol, yunantaxusin and taxusin, debenzoyl-2-acyl paclitaxel derivatives, benzoate paclitaxel derivatives, sulfonated 2′-acryloyltaxol; sulfonated 2′-O-acyl acid paclitaxel derivatives, C18-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, C7 taxane derivatives, C10 taxane derivatives, 2-debenzoyl and 2-acyl paclitaxel derivatives, taxane analogues bearing new C2 and C4 functional groups, n-acyl paclitaxel analogues, 10-deacetyl taxol B, and 10-deacetyl taxol, benzoate derivatives of taxol, 2-aroyl-4-acyl paclitaxel analogues, ortho-ester paclitaxel analogues, and deoxy paclitaxel and deoxy paclitaxel analogues.

In one aspect, the anti-microtubule agent is a taxane having the formula (C1): 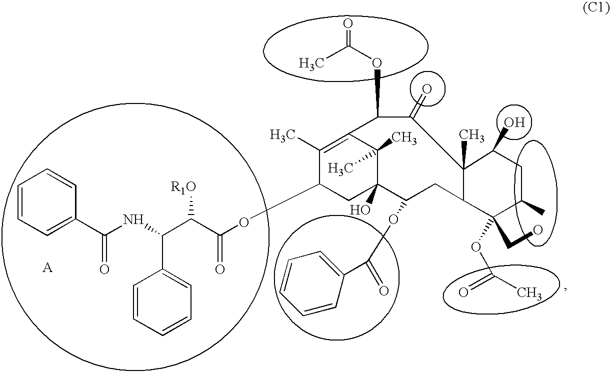
where the gray-highlighted portions may be substituted and the non-highlighted portion is the taxane core. A side-chain (labeled “A” in the diagram) is desirably present in order for the compound to have good activity as an anti-microtubule agent. Examples of compounds having this structure include paclitaxel (Merck Index entry 7117), docetaxol (TAXOTERE, Merck Index entry 3458, Aventis Pharma S.A., France), and 3′-desphenyl-3′-(4-ntirophenyl)-N-debenzoyl-N-(t-butoxycarbonyl)-10-deacetyltaxol.

In certain embodiments, suitable taxanes such as paclitaxel and its analogues and derivatives are disclosed in U.S. Pat. No. 5,440,056 as having the structure (C2): 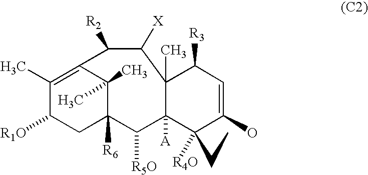
wherein X may be oxygen (paclitaxel), hydrogen (9-deoxotaxol or 9-deoxy derivatives, which may be further substituted to yield taxanes such as 7-deoxy-9-deoxotaxol, 10-desacetoxy-7-deoxy-9-deoxotaxol,), thioacyl, or dihydroxyl precursors; R₁ is selected from paclitaxel or taxotere side chains or an alkanoyl of the formula (C3) 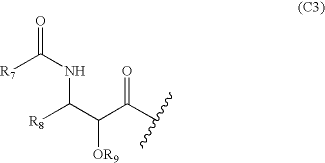
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy to yield taxanes that include in some cases with further substitution: 10-deacetyltaxol, 10-desacetoxy-11,12-dihydrotaxol-10,12(18)-diene derivatives, 10-deacetyl taxol A, 10-deacetyl taxol B; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In certain embodiments, the paclitaxel analogues and derivatives useful as anti-microtubule agents in the present invention are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

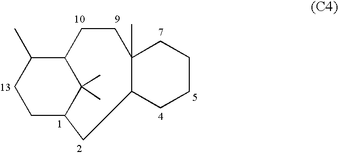

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based anti-microtubule agent useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

d) Fibrosing Agents

In certain embodiments, the therapeutic agent may be a fibrosing agent that induces fibrosis or scarring. When used in association with a device, it promotes cellular proliferation, thereby enhances fibrosis and adhesion between the device and the surrounding tissue. In addition, the fibrosing agent can be used to treat aneurysms and to stabilize vulnerable plaque from an arterial lumen.

Therapeutic agents that promote fibrosis or scarring can do so through one or more mechanisms including: inducing or promoting angiogenesis, stimulating migration or proliferation of connective tissue cells (such as fibroblasts, smooth muscle cells, vascular smooth muscle cells), inducing ECM production, and/or promoting tissue remodeling. In addition, numerous therapeutic agents described in this invention will have the additional benefit of also promoting tissue regeneration (the replacement of injured cells by cells of the same type).

Fibrosing agents are described, e.g., in the U.S. patent application entitled “Medical Implants and Fibrosis-Inducing Agents,” filed Nov. 20, 2004 (U.S. Ser. No. 10/986,230) and in the U.S. patent application entitled “Compositions and Methods for Treating Diverticular Disease,” filed May 12, 2005 (U.S. Ser. No. 11/129,763), both applications are incorporated by reference in their entireties. Exemplary fibrosing agents include, but are not limited to, silk (such as silkworm silk, spider silk, recombinant silk, raw silk, hydrolyzed silk, acid-treated silk, and acylated silk), talc, chitosan, polylysine, fibronectin, bleomycin or an analogue or derivative thereof, a fibrosing agent can be a connective tissue growth factor (CTGF), metallic beryllium or an oxide thereof, copper, saracin, silica, crystalline silicates, quartz dust, talcum powder, ethanol, a component of extracellular matrix, collagen, fibrin, fibrinogen, poly(ethylene terephthalate), poly(ethylene-co-vinylacetate), N-carboxybutylchitosan, an RGD protein, a polymer of vinyl chloride, cyanoacrylate, crosslinked poly(ethylene glycol)-methylated collagen, an inflammatory cytokine, TGFβ, PDGF, VEGF, TNFα, NGF, GM-CSF, IGF-a, IL-1, IL-8, IL-6, a growth hormone, a bone morphogenic protein, a cell proliferative agent, dexamethasone, isotretinoin, 17-β-estradiol, estradiol, diethylstibesterol, cyclosporine a, all-trans retinoic acid or an analogue or derivative thereof, wool (including animal wool, wood wool, and mineral wool), cotton, bFGF, polyurethane, polytetrafluoroethylene, poly(alkylcyanoacrylate), activin, angiopoietin, insulin-like growth factor (IGF), hepatocyte growth factor (HGF), a colony-stimulating factor (CSF), erythropoietin, an interferon, endothelin-1, angiotensin 11, bromocriptine, methylsergide, fibrosin, fibrin, an adhesive glycoprotein, proteoglycan, hyaluronan, secreted protein acidic and rich in cysteine (SPaRC), a thrombospondin, tenacin, a cell adhesion molecule, an inhibitor of matrix metalloproteinase, a tissue inhibitor of matrix metalloproteinase, methotrexate, carbon tetrachloride, and thioacetamide.

e) Anti-Inflammatory Agents

In certain embodiments, the therapeutic agent may be an anti-inflammatory agent that inhibits inflammation. Anti-inflammatory agents may be used individually or in combination with one or more of the therapeutic agents described herein.

Representative examples of anti-inflammatory agents are described in U.S. 2005/0004098 A1 and include aceclofenac, acemetacin, e-acetamidocaproic acid, acetaminophen, acetaminosalol, acetanilide, acetylsalicylic acid (aspirin), S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzitramide, benzpiperylon, benzydamine, benzylmorphine, bermoprofen, betamethasone, bezitramide, .alpha.-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butophanol, carbamazepine, carbiphene, carprofen, carsalam, celecoxib, chlorobutanol, chloroprednisone, chlorthenoxazin, choline magnesium trisalicylate, choline salicylate, cinchophen, cinmetacin, cinnoxicam, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide, cyclazocine, deflazacort, dehydrotestosterone, deracoxib, desomorphine, desonide, desoximetasone, dexamethasone, dexoxadrol, dextromoramide, dextropropoxyphene, dezocine, diamorphone, diampromide, diclofenac, difenamizole, difenpiramide, diflorasone, diflucortolone, diflunisal, difluprednate, dihydrocodeine, dihydrocodeinone enol acetate, dihydrocodeine phosphate, dihydromorphine, dihydroxyaluminum acetylsalicylate, dimenoxadol, dimepheptanol, dimethylthiambutene, dioxaphetyl butyrate, diphenhydramine hydrochloride, dipipanone, diprocetyl, dipyrone, ditazol, dl-chlorpheniramine maleate, droxicam, emorfazone, enfenamic acid, enoxolone, epirizole, eptazocine, etersalate, ethenzamide, ethoheptazine, etodolac, ethoxazene, ethoheptazine, ethylmethylthiambutene, ethylmorphine, etodolac, etofenamate, etonitazene, etoricoxib, eugenol, felbinac, fenbufen, fenchlofenac, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocortolone, fluoresone, fluorometholone, fluperolone, flupirtine, fluprednidene, fluprednisolone, fluprofen, fluproquazone, flurandrenolide, flurbiprofen, fluticasone, formocortal, fosfosal, furofenac, gentisic acid, glafenine, glucametacin, glycol salicylate, guaiazulene, halcinonide, halobetasol, halometasone, haloprednone, heroin, hydrocodone, hydrocortamate, hydrocortisone, hydromorphone, hydroxypethidine, ibufenac, ibuprofen, ibuproxam, imidazole salicylate, indomethacin, indoprofen, isofezolac, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, lysozyme chloride, mazipredone, meclofenamic acid, medrysone, mefenamic acid, meloxicam, meperidine, meprednisone, meptazinol, mesalamine, metazocine, methadone, methotrimeprazine, methylephedrine hydrochloride, methylprednisolone, methylsalicylate, metiazinic acid, metofoline, metopon, miroprofen, mofebutazone, mofezolac, mometasone, morazone, morphine, morphine hydrochloride, morphine sulfate, morpholine salicylate, myrophine, nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, noscapine, olsalazine, opium, oxaceprol, oxametacine, oxaprozin, oxipinac, oxycodone, oxymorphone, oxyphenbutazone, papaveretum, paramethasone, paranyline, parecoxib, parsalmide, pentazocine, perisoxal, phenacetin, phenadoxone, phenomorphan, phenazocine, phenazopyridine hydrochloride, phenocoll, phenoperidine, phenopyrazone, phenyl acetylsalicylate, phenylbutazone, phenylpropanolamine hydrochloride, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, rofecoxib, salacetamide, salicin, salicylamide, salicylamide o-acetic acid, salicylic acid, salicylsulfuric acid, salsalate, salverine, serratiopeptidase, simetride, sudoxicam, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaprofenic acid, tiaramide, tilidine, tinoridine, tiopinac, tioxaprofen, tixocortol, tolfenamic acid, tolmetin, tramadol, triamcinolone, tropesin, valdecoxib, viminol, xenbucin, ximoprofen, zaltoprofen, zidometacin, and zomepirac.

Other types of anti-inflammatory agent may be steroids, such as, for example, alclometasone, amcinonide, betamethasone, betamethasone 17-valerate, clobetasol, clobetasol propionate, clocortolone, cortisone, dehydrotestosterone, deoxycorticosterone, desonide, desoximetasone, dexamethasone, dexamethasone 21-isonicotinate, diflorasone, fluocinonide, fluocinolone, fluorometholone, flurandrenolide, fluticasone, halcinonide, halobetasol, hydrocortisone, hydrocortisone acetate, hydrocortisone cypionate, hydrocortisone hemisuccinate, hydrocortisone 21-lysinate, hydrocortisone sodium succinate, isoflupredone, isoflupredone acetate, methylprednisolone, methylprednisolone acetate, methylprednisolone sodium succinate, methylprednisolone suleptnate, mometasone, prednicarbate, prednisolone, prednisolone acetate, prednisolone hemisuccinate, prednisolone sodium phosphate, prednisolone sodium succinate, prednisolone valerate-acetate, prednisone, triamcinolone, and triamcinolone acetonide.

The anti-inflammatory agent may be an analgesic, such as, for example, alfentanil, allylprodine, alphaprodine, anileridine, benzylmorphine, bezitramide, buprenorphine, butorphanol, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, morphine, myrophine, nalbuphine, nalorphine, narceine, nicomorphine, norlevorphanol, normethadone, normorphine, norpipanone, opium, oxycodone, oxymorphone, papaveretum, pentazocine, phenadoxone, phenazocine, phenomorphan, phenoperidine, piminodine, piritramide, proheptazine, promedol, properidine, propiram, propoxyphene, sufentanil, tilidine, and tramadol.

The anti-inflammatory agent may be an NSAID, such as salicylic acid derivatives (such as salicylic acid, acetylsalicylic acid, methyl salicylate, diflunisal, olsalazine, salsalate, sulfasalazine and the like), indole and indene acetic acids (such as indomethacin, etodolac, sulindac and the like), fenamates (such as etofenamic, meclofenamic, mefenamic, flufenamic, niflumic and tolfenamic acids and the like), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin, zomepirac and the like), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, naproxen sodium, oxaprozin, pirprofen, pranoprofen, suprofen, tiaprofenic acid, tioxaprofen and the like), enolic acids (such as the oxicam derivatives ampiroxicam, cinnoxicam, droxicam, lomoxicam, meloxicam, piroxicam, sudoxicam and tenoxicam, and the pyrazolone derivatives aminopyrine, antipyrine, apazone, dipyrone, oxyphenbutazone, phenylbutazone and the like), para-aminophenol derivatives (such as acetaminophen and the like), alkanones (such as nabumetone and the like), nimesulide, and proquazone.

The anti-inflammatory agent may be a selective COX-2 inhibitor. A selective COX-2 inhibitor is a compound that selectively inhibits cyclooxygenase-2 (COX-2) activity.

(C) Additional Polymers

In certain embodiments, the polymeric coating composition of the present invention may further comprise one or more polymers in addition to the diblock copolymer. The additional polymers include bioerodable and non-bioerodable polymers.

In particular, the diblock copolymer can be formulated with additional polymers to provide polymeric coating materials having desirable physio-chemical properties suitable for a variety of medical devices. For example, physio-chemical properties such as hydrophilicity, swellability, viscosity, bioerodability, viscoelasticity and mechanical strength of the polymeric coating composition can be further modulated by combining the diblock copolymer with an additional polymer.

In certain embodiments, the additional polymer is a bioerodable polymer, for example, polyethylene glycol (PEG). Suitable PEGs include, but are not limited to, those with molecular weight of 200, 300, 400, 1000, 1450, 1500, 2000, 3000, 3350, 4000, 6000, 8000,10,000, 20,000, and 35000. Available commercial PEG products may be used with the present invention are, e.g., SIGRAMSA-ALDRICH, product numbers 95904 (MW 35004500), 81253 (MW 6000), 81255 (MW 6000), 89510 (MW 7000-9000), 81268 (MW 7000-9000), P2139 (MW 8000), P5413 (MW 8000), P4463 (MW 8000), P5667 (MW 10000), 92897 (MW 8500-11500), 95172 (16000-24000), and 94646 (35000).

In certain embodiments, the diblock copolymer helps to stabilize and strengthen a PEG-based polymeric coating composition. For example, the diblock copolymer and PEG can be combined at a weight ratio of about 0.3:3 to 0.7:3; or 0.6:3 to 0.8:3; or, 1:4,1:5, 1:6, 1:7, 1:8, or 1:9. □As demonstrated in Example 7, polymeric coating compositions thus formulated adhere to the needles for longer period of time in aqueous or tissue-like environment, than a PEG coating composition not reinforced with a diblock copolymer.

In other embodiments, the polymeric coating composition further comprises a non-bioerodable (also referred to as “biostable”) polymer. The biostable polymers are typically not water soluble or swellable, nor do they undergo hydrolytic degradation in vivo. They may harden and stabilize other components of the coating, without interfering with the character of the outer surface of the coating.

Examples of non-bioerodable polymers include acrylates, urethanes, polycarbonates, polyamides, polyesters and polyimides, cellulose ester polymers and copolymers, insoluble polyurethanes, polyvinyl chloride, polyamides, acrylate polymers and copolymers, ethylenevinylacetate copolymers, vinylpyrrolidoneethylacetate copolymers, acetal polymers and copolymers, silicone polymers and copolymers, polyesters, polyimides and copolymers, polybutadiene, polyisoprene and polyetherimides, poly(styrene-isobutylene-styrene), poly(styrene-isoprene-styrene), poly(styrene-butadiene-styrene), polystyrene, and alkylated polyvinylpyrrolidone.

(D) Other Additional Components

In certain embodiments, the polymeric coating compositions of the present invention may comprise other components in addition to a diblock copolymer, one or more therapeutic agents, and another polymer. Such additional components include, but are not limited to, buffers, solvents, colorants (e.g., Gentian Violet (Hucker Formula) and/or dimethylmethylene blue), surfactants (e.g., Tween 80, such as 1.00% w/w Tween 80 aq.), and other biocompatible components.

2) Insertable Medical Devices

A “medical device” or “device” generally refers to any insertable or implantable device for purpose of infusion, monitoring, maintaining a bodily passageway, occluding a passageway (e.g., an aneurysm), preventing surgical adhesion, and the like. Medical devices having various configurations and functions are contemplated within the scope of the present invention. Such devices include, but are not limited to, sensors (e.g., implantable glucose monitoring devices), pumps (e.g., implantable insulin pumps), stents, stent graft, heart valves, cardiac pacemakers, implantable cardioverter defibrillators, grafts (e.g., vascular grafts), ear, nose, or throat implants, urological implants, endotracheal or tracheostomy tubes, CNS shunts, orthopedic implants, ocular implants, pacemaker leads (e.g., silicone and polyurethane pacemaker leads), tubes (e.g., gastroenteric, drain, nasogastric, and endotracheal tubes), shunts (e.g., arteriovenous and hydrocephalous shunts), and deep brain stimulation (DBS) systems. Additional medical devices that can be coated with the polymeric coating composition include insertable devices such as needles and catheters, meshes suitable for wrapping an implanted medical device or an anatomical surface, and injectable microparticles (e.g., those comprise silk).

(A) Needles and Catheters

Without limiting the scope of the invention, insertable or implantable devices may include devices inserted into tissue, e.g., needles, or devices inserted into vessels or cavities, e.g., catheters. Examples of needles are an infusion set or device, a peripheral venous needle, an indwelling infusion needle, a butterfly needle, a subcutaneous access device, an insulin pump needle or a patient controlled analgesia (PCA) pump needle, and needles for fluid administration, amniocenteses, and biopsy. Examples of catheters are a peripheral venous catheter, an arterial catheter, a central venous catheter (CVC), a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, and a spinal or epidural catheter.

Such devices may be used, for example, to introduce various materials such as nutrients or therapeutic agents into patients, or to drain material from a patient (e.g., central nervous catheter containing an anti-infective drug, e.g., 5-fluorouracil and/or methotrexate).

In certain embodiments, only a portion of the device is inserted into the body of the patient and a portion of which protrudes outside of the body. In other embodiments, the device may be wholly implanted inside of the body of the patient, e.g., completely beneath the skin surface.

In certain embodiments, the present invention provides a needle or catheter coated with a polymeric coating composition, the polymeric coating composition comprising a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In certain embodiments, the weight ratio of the X block and Y block is about 65:35, 60:40, 55:45 or 50:50.

In certain embodiments, the X block is a polyether comprising alkylene oxide residues. The Y block is a polyester comprising hydroxy acid residues, as defined herein. Examples of the hydroxy acid include, but are not limited to, lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, and 1,4-dioxane-2-one and 1,5-dioxepan-2-one.

In one embodiment, the X block is poly(ethylene oxide), and the Y block comprises lactide residues. Preferably, the X block further comprises a terminal alkyl moiety, e.g., a methyl group. The X block, e.g., MePEG, has a molecular weight of at least 3,500. In one embodiment, the X block has a molecular weight of at least about 5,000. In other embodiments, the X block has a molecular weight of at least 6,500, at least 8,000, or at least 10,000.

In one embodiment, the diblock copolymer is MePEG-PDLLA (60:40) with a molecular weight of about 5,000.

In a further embodiment, the polymeric coating composition further comprises a polymer. In certain embodiments, the polymer is PEG. For example, the PEG has a molecular weight of 200, 300, 400, 1,000, 1,450, 1,500, 2,000, 3,000, 3,350, 4,000, 6,000, 8,000, 10,000, 20,000, and 35,000. More preferably, the PEG has a molecular weight of 3,500, 8,000,10,000, 20,000, 30,000 or 35,000.

In certain embodiments, the weight ratio of the diblock copolymer to PEG is between about 1:9 to 1:3. In other embodiments, the weight ratio of the diblock copolymer to PEG is between about 1:8 and 1:4. Preferably, the weight ratio is 1:5. The diblock copolymer reinforces the integrity and stability of the polymeric coating composition such that it adheres to the needle for an extended period of time. In certain embodiments, the polymeric coating composition remains firmly adhered to the needle for several hours or days in a tissue-like environment (e.g., an aqueous gelatin gel), compared to about an hour for PEG alone.

In certain embodiments, the polymeric coating composition further comprises a therapeutic agent. Suitable therapeutic agents include those that inhibit protein absorption or fibrotic tissue growth on the needle or catheter. Examples of the therapeutic agents include but are not limited to: one or more anti-fibrotic agents, anti-infective agents and anti-cancer agents, as described herein.

In certain embodiments, the anti-infective agents may be present (by weight) from about 0.1% to 50%, from about 0.5% to 30%, or from about 3% to 20% of the total weight of the polymeric coating composition. Examples of anti-infective agents include one or more of 2-bromo-2-nitropropane-1,3-diol (e.g., BRONOPOL), Irgasan (TRICLOSAN), polyhexanide (also known as polyhexamethylene biguanide) (e.g., VANTOCIL IB, COSMOCIL CQ, or BAQUACIL), benzalkonium chloride, benzethonium chloride, cetylpyradinium chloride, stearalkonium chloride, phenol, cresol, aminophenol, iodine, iodide, 8-hydroxyquinolone, chlorhexidine, anthracyclines (e.g., doxorubicin and mitoxantrone), fluoropyrimidines (e.g., 5-fluorouracil (5-FU)), folic acid antagonists (e.g., methotrexate), podophylotoxins (e.g., etoposide), camptothecins, hydroxyureas, and platinum complexes (e.g., cisplatin), and/or analogs or derivatives thereof. Other suitable anti-infective agents are as described herein.

In other embodiments, the anti-fibrotic agents or the anti-cancer agents may be present from 0.01 to 8.0%, from about 0.5 to 5.5% or from about 1.0 to 10% of the total weight of the polymeric coating composition. In other embodiments, an anti-infective agent can be combined with an anti-fibrotic agent and/or an anti-cancer agent.

Examples of anti-fibrotic agents include, but are not limited to, doxorubicin, mitoxantrone, TAXOTERE, vinblastine, tubercidin, paclitaxel, and analogues and derivatives thereof, podophyllotoxins (e.g., etoposide), immunomodulators (e.g., sirolimus and everolimus). Other suitable anti-fibrotic agents are as described herein.

The needles or catheter thus coated can maintain patency for a period of several days, a week or 10 days, compared to several hours or days for uncoated needles.

(B) Meshes

In other embodiments, the device may comprise or be in the form of a mesh. A mesh, as used herein, is a material composed of a plurality of fibers or filaments (i.e., a fibrous material), where the fibers or filaments are arranged in such a manner (e.g., interwoven, knotted, braided, overlapping, looped, knitted, interlaced, intertwined, webbed, felted, and the like) so as to form a porous structure.

Typically, a mesh is a pliable material, such that it has sufficient flexibility to be wrapped around a device or the external surface of a body passageway or cavity. In one embodiment, the mesh is used as a component of an intraluminal device (e.g., a vascular stent). In other embodiments, the mesh may be used as a perivascular wrap, which is placed into contact with (e.g., wrapped around) all or a portion of the external surface of a body passageway, such as a blood vessel, as part of a vascular surgical procedure. In certain aspects, the mesh may be sufficiently pliable so as to be capable of being wrapped around the external surface of a body passageway or cavity, or a portion thereof. The mesh may also be capable of providing support to the structure (e.g., the vessel or cavity wall) thereof. In certain aspects, the mesh may be adapted to release a therapeutic agent. More specifically, the mesh may be coated with a drug-loaded coating material.

In one embodiment, the present invention provides a mesh coated with a polymeric coating composition, the polymeric coating composition comprising a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In certain embodiments, the X block is a polyether comprising alkylene oxide residues. The Y block is a polyester comprising hydroxy acid residues, as defined herein. Examples of the hydroxy acid include, but are not limited to, lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one, and 1,5-dioxepan-2-one.

In one embodiment, the X block is poly(ethylene oxide), and the Y block comprises lactide residues. Preferably, the X block further comprises a terminal alkyl moiety, e.g., a methyl group. The X block, e.g., MePEG has a molecular weight of at least 3,500. In one embodiment, the X block has a molecular weight of at least about 5,000. In other embodiments, the X block has a molecular weight of at least 6,500, at least 8,000 or at least 10,000.

In certain embodiments, the weight ratios of the X blocks to Y blocks are about from 50:50 to 10:90. In one embodiment, the diblock copolymer is MePEG-PDLLA (20:80) with a molecular weight of about 5,000.

In other embodiments, the polymeric coating composition further comprises one or more therapeutic agents. The therapeutic agent comprises from about 2% to 25%, from about 5% to 20%, or from about 8% to 15% of the polymeric coating composition.

In certain embodiments, the polymeric coating composition comprises an anti-fibrotic agent. In one embodiment, the anti-fibrotic agent is paclitaxel. In other embodiment, the anti-fibrotic agent is chlorpromazine. In another embodiment, the anti-fibrotic agent is mycophenolic acid. Other suitable anti-fibrotic agents are as described herein.

In one embodiment, the present invention provides a mesh coated with MePEG-PDLLA (20:80) incorporating 5% paclitaxel, wherein MePEG has a molecular weight of about 5,000.

In certain embodiments, the polymeric coating composition comprises a fibrosing agent. In one embodiment, the fibrosis agent is an arterial wall irritant, such as silk, talcum powder, copper, saracin, silica, crystalline silicates, and quartz dust.

In a further embodiment, the polymeric coating composition further comprises a polymer. In certain embodiments, the polymer is PEG. For example, the PEG has a molecular weight of 200, 300, 400, 1,000,1,450, 1,500, 2,000, 3,000, 3,350, 4,000, 6,000, 8,000, 10,000, 20,000, and 35,000. More preferably, the PEG has a molecular weight of 3,500, 8,000,10,000, 20,000, 30,000 or 35,000. In one embodiment, the composition may include a PEG-based surgical sealant such as COSEAL® Surgical Sealant from Angiotech Pharmaceuticals (US), Inc. (North Bend, Wash.).

It is generally preferred that the coated mesh should not invoke biologically-detrimental inflammatory or toxic response, should be capable of being fully metabolized in the body, have an acceptable shelf life (of about at least one year or more), and be easily sterilized. Accordingly, in certain embodiments, the present invention provides a mesh of a bioerodable material combined with the bioerodable polymeric coating composition (with or without a therapeutic agent). Such a device, whether used alone or in conjunction with another implantable device, is expected to have enhanced biocompatibility. In particular, the polymeric coating composition is suited for coating mesh materials of different forms, on account of its tunable mechanical strength and viscoelasticity.

Mesh materials may take a variety of forms. For example, the mesh may be in a woven, knit, or non-woven form, and may include fibers or filaments that are randomly oriented relative to each other or that are arranged in an ordered array or pattern. In one embodiment, for example, a mesh may be in the form of a fabric, such as a knitted, braided, crocheted, woven, non-woven (e.g., a melt-blown or wet-laid), or webbed fabric. In one embodiment, a mesh may include a natural or synthetic biodegradable polymer that may be formed into a knit mesh, a weave mesh, a sprayed mesh, a web mesh, a braided mesh, a looped mesh, and the like. Preferably, a mesh or wrap has intertwined threads that form a porous structure, which may be, for example, knitted, woven, or webbed. The structure and properties of the mesh used in a device depend on the application and the desired mechanical (i.e., flexibility, tensile strength, and elasticity), degradation properties, and the desired loading and release characteristics for the selected therapeutic agent(s). Factors that affect the flexibility and mechanical strength of the mesh include, for example, the porosity, fabric thickness, fiber diameter, polymer composition (e.g., type of monomers and initiators), process conditions, and the additives that are used to prepare the material.

Flexible mesh materials are typically in the form of flexible woven or knitted sheets having a thickness ranging from about 25 microns to about 3000 microns; preferably from about 50 to about 1000 microns. Mesh materials for use in the practice of the invention typically range from about 100 to 400 microns in thickness.

Typically, the mesh possesses sufficient porosity to permit the flow of fluids through the pores of the fiber network and to facilitate tissue ingrowth. Generally, the interstices of the mesh should be wide enough apart to allow light visible by eye, or fluids, to pass through the pores. However, materials having a more compact structure also may be used. The flow of fluid through the interstices of the mesh may depend on a variety of factors, including, for example, the stitch count or thread density. The porosity of the mesh may be further tailored by, for example, filling the interstices of the mesh with another material (e.g., particles or polymer) or by processing the mesh (e.g., by heating) in order to reduce the pore size and to create non-fibrous areas. Fluid flow through the mesh of the invention can vary depending on the properties of the fluid, such as viscosity, hydrophilicity/hydrophobicity, ionic concentration, temperature, elasticity, pseudoplasticity, particulate content, and the like. The interstices of the mesh can be large enough so as to not prevent the release of impregnated or coated therapeutic agent(s) from the mesh, and the interstices preferably do not prevent the exchange of tissue fluid at the application site.

The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length). Depending on the application, the fibers may have a monofilament or a multifilament construction.

The mesh may include fibers that are of same dimension or of different dimensions, and the fibers may be formed from the same or different types of biodegradable polymers. Woven materials, for example, may include a regular or irregular array of warp and weft strands, and may include one type of polymer in the weft direction and another type (having the same or a different degradation profile from the first polymer) in the warp direction. The degradation profile of the weft polymer may be different from or the same as the degradation profile of the warp polymer. Similarly, knit materials may include one or more types (e.g., monofilament, multi-filament) and sizes of fibers, and may include fibers made from the same or from different types of biodegradable polymers.

The structure of the mesh (e.g., fiber density and porosity) may impact the amount of polymeric coating composition coated thereon and the therapeutic agent that may be loaded. For example, a fabric having a loose weave characterized by a low fiber density and high porosity can have a lower thread count, resulting in a reduced total fiber volume and surface area. As a result, the amount of agent that may be loaded into or onto, with a fixed polymeric coating composition:therapeutic agent ratio, the fibers can be lower than for a fabric having a high fiber density and lower porosity.

The device may include multiple mesh materials in any combination or arrangement. For example, a portion of the device may be a knitted material and another portion may be a woven material. In another embodiment, the device may more than one layer (e.g., a layer of woven material fused to a layer of knitted material or to another layer of the same type or a different type of woven material). In some embodiments, multi-layer constructions (e.g., device having two or more layers of material) may be used, for example, to enhance the performance properties of the device (e.g. for enhancing the rigidity or for altering the porosity, elasticity, or tensile strength of the device) or for increasing the amount of drug loading.

The mesh may be formed of or include a polymer. The polymer may be a biodegradable or a non-biodegradable polymer, or a combination thereof.

Biodegradable compositions that may be used to prepare the mesh include polymers that comprise albumin, collagen, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and 6-caprolactone, copolymers of lactide and 1,4-dioxane-2-one, polymers and copolymers that include one or more of the residue units of the monomers D-lactide, L-lactide, D,L-lactide, glycolide, δ-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). These compositions include copolymers of the above polymers as well as blends and combinations of the above polymers (see, generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). In one aspect, the mesh includes a biodegradable or resorbable polymer that is formed from one or more monomers selected from the group consisting of lactide, glycolide, e-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid), poly(L-lactide)/poly(D,L-lactide) blends or copolymers of L-lactide and D,L-lactide. In yet another aspect, the polymer includes a copolymer of lactide and ε-caprolactone. In yet another aspect, the polymer includes a polyester (e.g., a poly(lactide-co-glycolide). For example, the mesh may be prepared (e.g., knitted) from fibers formed from a copolymer of lactide and glycolide (e.g., PLGA)). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide). Other examples of biodegradable materials include polyglactin, polyglycolic acid, autogenous, heterogenous, and xenogeneic tissue (e.g., pericardium or small intestine submucosa), and oxidized, regenerated cellulose. These meshes can be knitted, woven or non-woven meshes. Other examples of non-woven meshes include electrospun materials.

Representative examples of non-biodegradable compositions for use in forming meshes include ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), polyolefins such as poly(ethylene) or poly(propylene), polyamides (e.g., nylon 6,6), poly(urethanes) (e.g. poly(ester urethanes), poly(ether urethanes), poly(carbonate urethanes), poly(ester-urea)), polyesters (e.g., PET, polybutyleneterephthalate, and polyhexyleneterephthalate), polyethers (poly(ethylene oxide), poly(propylene oxide), poly(ethylene oxide)-poly(propylene oxide) copolymers, diblock and triblock copolymers, poly(tetramethylene glycol)), silicone containing polymers and vinyl-based polymers (polyvinylpyrrolidone, poly(vinyl alcohol), poly(vinyl acetate phthalate), poly(styrene-co-isobutylene-co-styrene), fluorine containing polymers (fluoropolymers) such as fluorinated ethylene propylene (FEP) or polytetrafluoroethylene (e.g., expanded PTFE).

Meshes which may be coated with a polymeric coating composition, include commercially available products, such as INTERCEED (Johnson & Johnson, Inc.), PRECLUDE (W. L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. In addition, Boston Scientific Corporation sells the TRELEX NATURAL Mesh, which is composed of a unique knitted polypropylene material. Ethicon, Inc. makes the absorbable VICRYL (polyglactin 910) meshes (knitted and woven) and MERSILENE Polyester Fiber Mesh. Dow Corning Corporation (Midland, Mich.) sells a mesh material formed from silicone elastomer known as SILASTIC Rx Medical Grade Sheeting (Platinum Cured). United States Surgical/Syneture (Norwalk, Conn.) sells a mesh made from absorbable polyglycolic acid under the trade name DEXON Mesh Products. Membrana Accurel Systems (Obernburg, Germany) sells the CELGARD microporous polypropylene fiber and membrane. Gynecare Worldwide, a division of Ethicon, Inc. sells a mesh material made from oxidized, regenerated cellulose known as INTERCEED TC7. Integra LifeSciences Corporation (Plainsboro, N.J.) makes DURAGEN PLUS Adhesion Barrier Matrix, which can be used as a barrier against adhesions following spinal and cranial surgery and for restoration of the dura mater. HYDROSORB Shield from MacroPore Biosurgery, Inc. (San Diego, Calif.) is a film for temporary wound support to control the formation of adhesions in specific spinal applications.

Other commercially available meshes include (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N. H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3,4 & 5 and HERNIAMESH plugs T1, T2, and T3 from Herniamesh USA, Inc. (Great Neck, N.Y.).

Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the polymeric coating composition may be applied only to the uncoated side of SEPRAMESH and not to the sodium hyaluronate/carboxymethylcellulose coated side.

(C) Injectable Microparticles

In another embodiment, the present invention provides an injectable formulation comprising microparticles, the microparticles being encapsulated in a polymeric coating composition, wherein the polymeric coating composition comprises a bioerodable diblock copolymer of Formula: X—Y (m:n) having a molecular weight of at least 7500, wherein, X is a hydrophilic poly(alkylene oxide) having a molecular weight of at least 3500, Y is a hydrophobic polyester, m represents a weight percentage of X based on a total weight of the diblock copolymer, and n represents a weight percentage of Y based on the total weight of the diblock copolymer, m+n=100.

In certain embodiments, the X block is a polyether comprising alkylene oxide residues. The Y block is a polyester comprising hydroxy acid residues, as defined herein.

In one embodiment, the X block is poly(ethylene oxide), and the Y block comprises lactide residues. Preferably, the X block further comprises a terminal alkyl moiety, e.g., a methyl group. The X block, e.g., MePEG has a molecular weight of at least 3,500. In one embodiment, the X block has a molecular weight of at least about 5,000. In other embodiments, the X block has a molecular weight of at least 6,500, at least 8,000 or at least 10,000.

In certain embodiments, the weight ratios of the X block to Y block are from 65:35 to 60:40. In one embodiment, the diblock copolymer is MePEG-PDLLA (60:40) with a molecular weight of MePEG being about 5,000.

“Microparticle” refers to a particle of microscopic size. Typically, the diameters of the microparticles (i.e., the distance spanning the widest point, or points, of the microparticle) are about 0.5 μm to 1,000 μm. Microparticles may have regular or irregular shapes.

The microparticles can be delivered to a desired location into a host, typically through injection. In certain embodiments, the microparticles are therapeutic. Examples of the therapeutic microparticles include arterial wall irritants, which promotes fibrosis formation. For example, the microparticles can be silk, talcum powder, chitosan copper, saracin, silica, crystalline silicates, quartz dust.

In other embodiments, microparticles can be a drug-delivery vehicle, comprising one or more therapeutic agents, as defined herein. Drug-loaded microparticles are well known in the art.

In certain embodiments, the microparticles have a preferred average diameter of at least about 0.5 μm, 1 μm, 5 μm, 10 μm, 20 μm, 50 μm or 100 μm, the optimal size being determined by the desired drug release properties and the application. In certain embodiments, the microparticles have a preferred average diameter of no more than about 5 μm, 10 μm, 20 μm, 50 μm, 100 μm, 150 μm, 250 μm, 500 μm, or 1,000 μm, the optimal size being determined by the desired drug release properties and the application.

In certain embodiments, the microparticles have a size distribution of 10-100 μm, 100-500 μm or 500-1,000 μm.

In certain embodiments, the injectable formulation comprises about equal amount of the microparticles and diblock copolymer by weight. In other embodiment, the microparticles are from about 90-95% of the weight of the diblock copolymer. In other embodiments, the microparticles are from about 85-92% of the weight of the diblock copolymer. In other embodiments, the microparticles are from about 80-93% of the weight of the diblock copolymer.

In a further embodiment, the injectable formulation further comprises a buffer. Buffers capable of maintaining a physiological pH are well known to one skilled in the art. In one embodiment, the injectable formulation comprises a pH 7.3 buffer.

According to the present invention, microparticles encapsulated in the polymeric coating composition provides an efficient delivery of the microparticles by injection. In an aqueous formulation, the polymeric coating composition swells to form a gel layer encasing the microparticles (either partially or fully). The gel layer is soft and deformable, which allows the microparticles to be injected through a small needle or catheter. Following injection through the opening of the needle or catheter, the gel-coated microparticles revert to their swollen sizes. The microparticles thus injected remain at the injection site without being pulled out by the retrieving needle, or diffusing from the application site. Over a period of time, the bioerodable polymeric coating composition erodes and exposes the microparticles.

In certain embodiments, the microparticles are silk particles. Silk is known for its fibrosis-inducing capability and has been used to provide adhesion between an implantable device and the surrounding tissue. Moreover, particulate silk formulation, with or without additional fibrosis-inducing agent, can be used to occlude an aneurysm.

Silk refers to a fibrous protein and may be obtained from a number of sources; typically spiders and silkworms. Typical silk contains about 75% of actual fiber, referred to as fibroin, and about 25% sericin, which is a gummy protein that holds the filaments together. Silk filaments are generally very fine and long—as much as 300-900 meters long. There are several species of domesticated silkworm that are used in commercial silk production, however, Bombyx mori is the most common, and most silk comes from this source. Other suitable silkworms include Philosamia ricini, Antheraea yamamai, Antheraea pernyi, and Antheraea mylitta. Spider silk is relatively more difficult to obtain, however, recombinant techniques hold promise as a means to obtain spider silk at economical prices (see, e.g., U.S. Pat. Nos. 6,268,169; 5,994,099; 5,989,894; and 5,728,810, which are exemplary only). Biotechnology has allowed researchers to develop other sources for silk production, including animals (e.g., goats) and vegetables (e.g., potatoes). Silk from any of these sources may be used in the present invention.

A commercially available silk protein is available from Croda, Inc., of Parsippany, N.J., and is sold under the trade names CROSILK LIQUID (silk amino acids), CROSILK 10,000 (hydrolyzed silk), CROSILK POWDER (powdered silk), and CROSILKQUAT (cocodiammonium hydroxypropyl silk amino acid). Another example of a commercially available silk protein is SERICIN, available from Pentapharm, LTD, a division of Kordia, BV, of the Netherlands. Further details of such silk protein mixtures can be found in U.S. Pat. No. 4,906,460, to Kim, et al., assigned to Sorenco. Silk useful in the present invention includes natural (raw) silk, degummed silk, hydrolyzed silk, and modified silk, i.e., silk that has undergone a chemical, mechanical, or vapor treatment, e.g., acid treatment or acylation (see, e.g., U.S. Pat. No. 5,747,015).

The silk used in the present invention may be in the form of particles (e.g., the silk may be in the form of a powder). Furthermore, the silk may have any molecular weight, where various molecular weights are typically obtained by the hydrolysis of natural silk, where the extent and harshness of the hydrolysis conditions determines the product molecular weight. For example, the silk may have an average (number or weight) molecular weight of 200 to 5,000. See, e.g., JP-B-59-29199 (examined Japanese patent publication) for a description of conditions that may be used to hydrolyze silk. Silk particles can also be obtained by freeze-milled from silk fibers or filaments directly.

3) Methods for Preparing Medical Devices Coated With Polymeric Coating Compositions

In one aspect, the present invention provides a method for preparing an insertable medical device that comprises a polymeric coating composition as described herein. The polymeric coating composition, with or without a therapeutic agent, can be combined with the device in a variety of ways.

In certain embodiments, the polymeric composition may be coated onto the entire device or a portion of the device using a method, such as by dipping, spraying, painting or vacuum deposition, and ink jet coating, that is appropriate for the particular type of device. In other embodiments, the polymeric coating composition may be incorporated into a device having channels, divets or voids opening to an outer surface of the device. “Coating”, as used herein, thus encompasses any process of applying the polymeric coating composition (with or without a therapeutic agent) to a surface of an insertable medical device, as defined herein. As noted above, the surface may be the entire or partial outer surface of the device, or a surface of any channel, divet or void in the body of the device.

In general, a coating composition for a medical device is characterized with physical properties that allow for the coating composition to crimp and expand without tearing or detaching from the device. Depending on the specific structure and function of the device, a coating composition can be selected based on factors including mechanical strength, hydrophilicity, viscosity, viscoelasticity, swellability, adhesion and the like.

a) Dip coating

Dip coating is one exemplary process that can be used to combine a polymeric coating composition (with or without a therapeutic agent) with a device. In one embodiment, the polymeric coating composition is dispersed in a solvent and is then coated onto an outer surface of the device. Dip coating is also suitable for incorporating the polymeric coating composition into devices having channels, divets and voids that open to an outer surface of the device.

In certain embodiments, an inert solvent may be selected to avoid dissolving the device. In other embodiments, a swelling solvent may be selected to swell the device to certain degrees. In yet other embodiments, a solvent may be selected to dissolve the device over time. Where a therapeutic agent is incorporated into the polymeric coating composition, a solvent may be selected to facilitate the dispersion of the therapeutic agent in the polymeric coating composition.

Coating with an Inert Solvent

In one embodiment, the solvent is an inert solvent for the device such that the solvent does not dissolve the medical device to any great extent and is not absorbed by the device to any great extent. The device can be immersed, either partially or completely, in the polymeric coating composition/solvent (with or without a therapeutic agent) dispersion for a specific period of time. The rate of immersion into polymer/solvent dispersion can be altered (e.g., 0.001 cm per sec to 50 cm per sec). The device can then be removed from the solution. The rate at which the device can be withdrawn from the dispersion can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the polymeric coating composition (with or without a therapeutic agent) being coated on the surface of the device.

Coating with a Swelling Solvent

In one embodiment, the solvent is one that will not dissolve the device but will be absorbed by the device. These solvents can thus swell the device to some extent. The device can be immersed, either partially or completely, polymer/solvent dispersion for a specific period of time (seconds to days). A therapeutic agent can optionally be suspended in the dispersion. The rate of immersion into the polymer/solvent dispersion can be altered (e.g., 0.001 cm per sec to 50 cm per sec). The device can then be removed from the dispersion. The rate at which the device can be withdrawn from the dispersion can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the polymeric coating composition being coated onto the surface of the device as well as the potential for the therapeutic agent being adsorbed into the medical device. The therapeutic agent may also be present on the surface of the device. The amount of surface-associated therapeutic agent may be reduced by dipping the coated device into a solvent for the therapeutic agent or by spraying the coated device with a solvent for the therapeutic agent.

Coating with a Solvent

In one embodiment, the solvent is one that will be absorbed by the device and that will dissolve the device. The device can be immersed, either partially or completely, in the polymer/solvent dispersion for a specific period of time (seconds to hours). A therapeutic agent can also be suspended in the dispersion. The rate of immersion into the polymeric coating composition/solvent dispersion can optionally be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). The therapeutic agent may also be present on the surface of the device. The amount of surface associated therapeutic agent may be reduced by dipping the coated device into a solvent for the therapeutic agent or by spraying the coated device with a solvent for the therapeutic agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

As noted, in any of the above dip-coating processes, a therapeutic agent can be suspended in the polymeric coating composition and solvent dispersion. The suspension can be prepared by choosing a solvent that can dissolve the polymer but not the therapeutic agentor a solvent that can dissolve the polymer and in which the therapeutic agent is above its solubility limit. In similar processes described above, a device can be dipped into the suspension of the therapeutic agent and polymeric coating composition/solvent such that the device is coated with a polymeric coating composition that has a therapeutic agent suspended within it.

b) Spray Coating

Spray coating is another coating process that can be used. In the spray coating process, a solution or dispersion of polymeric coating composition, with or without a therapeutic agent, is nebulized and directed to the device to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the polymeric coating composition is formulated in a solvent for the therapeutic agent and is then sprayed onto the device. In certain embodiments, the polymeric coating composition/solvent dispersion can be sprayed onto the entire outer surface of the device. In other embodiments, a mask can be used so that only parts of a device are sprayed with the polymeric coating composition/solvent dispersion.

Spraying with an Inert-Solvent

In one embodiment, the solvent is an inert solvent for the device such that the solvent does not dissolve the medical device to any great extent and is not absorbed by the device to any great extent. The device can be spray coated, either partially or completely, using a polymeric coating composition/solvent composition. The rate of spraying of the polymeric coating composition/solvent dispersion can be altered (e.g., 0.001 mL per sec to 10 mL per sec) to ensure that a good coating of polymeric coating composition is obtained. The coated device can be air-dried. The spray coating process can be repeated one or more times depending on the specific application. The device can be dried under vacuum to reduce residual solvent levels. This process will result in the polymeric coating composition (with or without a therapeutic agent) being coated on the surface of the device.

Sparying with a Swelling Solvent

In one embodiment, the solvent is one that will not dissolve the device but will be absorbed by the device. These solvents can thus swell the device to some extent. The device can be spray coated, either partially or completely using a polymeric coating composition/solvent composition (with or without a therapeutic agent). The rate of spraying of the polymeric coating composition/solvent dispersion can be altered (e.g., 0.001 mL per sec to 10 mL per sec) to ensure that a good coating of the polymeric coating composition is obtained. The coated device can be air-dried. The spray coating process can be repeated one or more times depending on the specific application. The device can be dried under vacuum to reduce residual solvent levels. This process will result in the polymeric coating composition being coated onto the surface of the device as well as the potential for the therapeutic agent being adsorbed into the medical device. The therapeutic agent may also be present on the surface of the device. The amount of surface-associated therapeutic agent may be reduced by dipping the coated device into a solvent for the therapeutic agent or by spraying the coated device with a solvent for the therapeutic agent.

Spraying with a Solvent

In one embodiment, the solvent is one that will be absorbed by the device and that will dissolve the device. The device can be spray coated, either partially or completely, using a polymeric coating composition/solvent composition (with or without a therapeutic agent). The rate of spraying of the polymeric coating composition/solvent solution can be altered (e.g., 0.001 mL per sec to 10 mL per sec) to ensure that a good coating of the polymeric coating composition is obtained. The coated device can be air-dried. The spray coating process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). In certain embodiment, a therapeutic agent may also be present on the surface of the device. The amount of surface associated therapeutic agent may be reduced by dipping the coated device into a solvent for the therapeutic agent or by spraying the coated device with a solvent for the therapeutic agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

As noted, in any of the above spray-coating processes, a therapeutic agent can be suspended in the polymeric coating composition and solvent dispersion. The suspension can be prepared by choosing a solvent that can dissolve the polymer but not the therapeutic agent, or a solvent that can dissolve the polymer and in which the therapeutic agent is above its solubility limit. In similar processes described above, a device can be dipped into the suspension of the therapeutic agent and polymeric coating composition/solvent such that the device is coated with a polymeric coating composition that has a therapeutic agent suspended within it.

In certain embodiments, a medical device may include a plurality of openings or reservoirs within its structure, each opening configured to house into which a polymeric coating composition (with or without a therapeutic agent) of the present invention can be incorporated. The reservoirs may be formed from divets or wells in the device surface or micropores or channels in the device body. The reservoirs may be formed, e.g., from voids or openings in the structure of the device. For example, a drug-loaded polymer coating composition described herein may be loaded into one or more of the reservoirs. The filled reservoir can function as a drug delivery depot which can release drug over a period of time dependent on the release kinetics of the drug from the polymer. In certain embodiments, the reservoir may be loaded with a plurality of layers, each layer including a different drug having a particular amount (dose) of drug in a polymeric coating composition, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

Methods for preparing exemplary insertable medical devices (i.e., needles or catheters, meshes, and injectable microparticle formulations) are provided below in greater detail. Such methods may be used for preparing other insertable medical devices when appropriate.

(A) Needles and Catheters

In one embodiment, the present invention relates to a method of coating an insertable needle or catheter, comprising: applying a polymeric coating composition described herein to the insertable needle or catheter.

In certain embodiments, the applying step comprises: (a) applying the polymeric coating composition prior to packaging the needle or catheter, and/or (b) coating the needle or catheter with a moistened swab or pad after removing the needle or catheter from its package prior to insertion.

In certain embodiments, the polymeric coating composition is mixed with one or more solvents prior to the application (or coating) step to form a pre-coating solution. The solvent may be selected from those that are able to dissolve or disperse the components of the polymeric coating composition (e.g., diblock copolymers, additional polymers, and/or therapeutical agents) and form a homogeneous pre-coating solution. In particular, suitable solvents include those that are compatible with the therapeutic agents present in polymeric coating compositions, and are appropriate for human use as residues in the coating. Examples of the solvents include one or more of the following: water, acetonitrile, methylethyl ketone (MEK), denatured ethanol, ethyl alcohol (ethanol), saline solution, normal saline solution, tetrahydrofuran (THF), isopropyl alcohol (isopropanol), other alcohols, amines, amides, 1,3-dioxalane, ketones, esters, cyclic compounds, glycols, carboxylic acids or aromatic solvents. In another exemplary embodiment, the solvent may be cyclohexanone, toluene, benzyl alcohol, dibutylphthalate, butanol, xylene and/or ethyl benzene.

One skilled in the art will recognize that the thickness of the coating is determined by the concentration of the polymeric coating composition in the pre-coating solution, as well as by the number of coatings applied. In an exemplary embodiment, the pre-coating solution may comprise from about 50% to about 99.95% or from about 70% to 99%, 70% to 80%, 80% to 90%, or 90% to about 98.8% solvent.

In one embodiment, the coating may be applied by spraying, dipping or wiping. In another exemplary embodiment, the coating may be manufactured using an extrusion process.

In a further aspect, the coated needles or catheters may be dried at an elevated temperature to allow the solvent to evaporate. For example, the coated needles or catheters can be dried by heating, e.g., an oven or a blow dryer, at a temperature of at least about 40° C., 40 to 100° C., 40 to 90° C., 40 to 60° C., or about 40, 50, 60, 70, 80 or 90° C. Persons skilled in the art will recognize that exposing the coated device to elevated temperatures can be used to remove solvent from the coating and/or to cure the coating. The time and temperature should be selected so as to accomplish the above efficiently and without exposing the coating composition to excessive heat that may damage one or more components in the coating or underlying substrate. In a further embodiment, a primer and/or a base coat can be applied prior to applying the polymeric coating composition. In such a multi-layer coating, the polymeric coating composition comprising the diblock copolymer is also referred to as a “top coat”. In certain embodiments, the primer layer comprises polyethylene-co-acrylic acid polymer, epoxy resin and/or polyurethane resin. The basecoat layer comprises at least one bioerodable polymer such as PEG, and/or at least one biostable polymer, as defined herein. The primer and the base coat cause the top coat to firmly adhere to the needles.

(B) Meshes

In one embodiment, the present invention provides a method of coating a mesh comprising: applying a polymeric coating composition described herein to a surface of the mesh. In certain embodiments, the polymeric coating composition can be applied to the surface of the mesh by: dipping the mesh into or spraying the mesh with the polymeric coating composition.

Typically, the polymeric coating composition is dissolved or dispersed in a solvent to form a pre-coating solution. Suitable solvents include, but are not limited to, one or more of the following: water, acetonitrile, methylethyl ketone (MEK), denatured ethanol, ethyl alcohol (ethanol), saline solution, normal saline solution, tetrahydrofuran (THF), isopropyl alcohol (isopropanol), other alcohols, amines, amides, 1,3-dioxalane, ketones, esters, cyclic compounds, glycols, carboxylic acids or aromatic solvents. In another exemplary embodiment, the solvent may be cyclohexanone, toluene, benzyl alcohol, dibutylphthalate, butanol, xylene, and ethyl benzene.

One skilled in the art will recognize that the thickness of the coating is determined by the concentration of the polymeric coating composition in the pre-coating solution, as well as by the number of coatings applied. In an exemplary embodiment, the pre-coating solution may comprise from about 50% to about 99% or from about 70% to 99%, 70% to 80%, 80% to 90% solvent.

In certain embodiments, the polymeric coating composition may further comprise a therapeutic agent, in particular, an anti-fibrotic agent, as described herein. In various embodiments, the anti-fibrotic agent is paclitaxel, chlorpromazine, or mycophenolic acid. The anti-fibrotic agent can be added directly to the pre-coating solution prior to the application step.

In other embodiments, the polymeric coating composition may comprise a fibrosing agent, as described herein. In one embodiment, the fibrosing agent is silk. The fibrosing agent can be suspended in the pre-coating solution prior to the application step.

In other embodiments, the method of coating a mesh further comprises allowing the solvent to evaporate. The dried polymeric coating composition-coated meshes have long shelf lives. They can be packed between two pieces of release liners and stored in a sealed package. Once exposed to an aqueous environment (e.g., tissue or tissue-like environment), the polymeric coating composition releases the therapeutic agent incorporated therein.

The mesh (or device used in conjunction with the mesh) may be made sterile either by preparing them under aseptic environment and/or they may be terminally sterilized using methods known in the art, such as gamma radiation or electron beam sterilization methods or a combination of both of these methods.

(C) Injectable Microparticles

In certain embodiments, the present invention provides a method of preparing an injectable microparticle formulation comprising: (a) mixing microparticles and a diblock copolymer in a solvent to provide a suspension, the diblock copolymer being represented by Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100; and (b) spray-drying the suspension to provided diblock copolymer-coated microparticles.

In certain embodiments, the diblock copolymer can be dissolved in the solvent prior to suspending the microparticles. Examples of the solvent include dichloromethane, THF and the like.

In other embodiment, the method further comprises vacuum drying the diblock-copolymer coated microparticles to remove the residual solvent.

In a further embodiment, the method comprises mixing the diblock copolymer-coated microparticles with a buffer.

B. Method of Using Medical Devices Coated with Polymeric Coating Compositions

In one aspect, the invention provides a method of using a medical device comprising:

(a) combining the medical device with a polymeric coating composition comprising a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100; and

(b) inserting the coated medical device into a subject.

In various embodiments, in terms of percentage, m is about 40-75, about 45-70, or about 50-65.

In various embodiments, the X block is a poly(ethylene oxide) and the Y block is a polylactide (PDLLA). The X block may further comprise a terminal alkyl group, e.g., a methyl.

In certain embodiments, the diblock copolymer of the polymeric coating composition is MePEG:PDLLA (60:40), wherein the MePEG has a molecular weight of about 5,000.

In a further embodiment, the polymeric coating composition further comprises another polymer. In certain embodiments, the polymer is PEG. For example, the PEG has a molecular weight of 200, 300, 400, 1000, 1,450,1,500, 2,000, 3,000, 3,350, 4,000, 6,000, 8,000,10,000, 20,000, or 35,000. More preferably, the PEG has a molecular weight of 3500, 8000,10,000, 20,000, 30,000 or 35,000.

In certain embodiments, the weight ratio of the diblock copolymer to PEG is between about 1:9 to 1:3. In other embodiments, the weight ratio of the diblock copolymer to PEG is between about 1:8 and 1:4. Preferably, the weight ratio is 1:5.

In certain embodiment, the polymeric coating composition further comprises a therapeutic agent. In one embodiment, therapeutic agent is an anti-infective agent. In certain embodiments, the anti-infective agents may be present from about 0.1% to 50%, from about 0.5% to 30% or from about 3% to 20% of the total weight of the polymeric coating composition. Examples of anti-infective agents include one or more of 2-bromo-2-nitropropane-1,3-diol (e.g., BRONOPOL), Irgasan (TRICLOSAN), polyhexanide (also known as polyhexamethylene biguanide) (e.g., VANTOCIL IB, COSMOCIL CQ, or BAQUACIL), benzalkonium chloride, benzethonium chloride, cetylpyradinium chloride, stearalkonium chloride, phenol, cresol, aminophenol, iodine, iodide, 8-hydroxyquinolone, and chlorhexidine. Other suitable anti-infective agents are as described herein.

The insertable medical device coated with the polymeric coating composition may reduce the incidence and/or severity of protein absorption and build up and/or the incidence and/or severity of infections occurring at or associated with the site of insertion of the device. Thus, in a related aspect, the present invention also provides a method for exending patency of an insertable medical device by coating the insertable medical device with the polymeric coating composition as described herein. In certain embodiments, the device is inserted and remains patent for at least about 5 days or longer, e.g. 5 to 10 days, 6 to 9 days, 7 to 8 days, 6 days, 7 days, 8 days, 9 days or 10 days.

In other embodiments, the therapeutic agent is an anti-fibrotic agent or an anticancer agent. The therapeutic agent may be present from 0.01 to 8.0%, from about 0.5 to 5.5% or from about 1.0 to 10% of the total weight of the polymeric coating composition. In other embodiments, an anti-infective agent can be combined with an anti-fibrotic agent and/or an anti-cancer agent.

Examples of the suitable therapeutic agents include, but are not limited to, doxorubicin, mitoxantrone, TAXOTERE, vinblastine, tubercidin, paclitaxel, and analogues and derivatives thereof, podophyllotoxins (e.g., etoposide), immunomodulators (e.g., sirolimus and everolimus). Other suitable anti-fibrotic agents are as described herein.

Methods of using two exemplary insertable medical devices (i.e., coated meshes and injectable microparticle formulations) are described in greater detail below.

1) Method of Using a Coated Mesh (or Film)

The films or meshes coated with polymeric coating compositions of the present invention may be used for a variety of indications, including, without limitation: (a) prevention or reduction of surgical adhesions between tissues following surgery (e.g., gyneacologic surgery, vasovasostomy, hernia repair, nerve root decompression surgery and laminectomy); (b) prevention or reduction of hypertrophic scars or keloids (e.g., resulting from tissue burns or other wounds); (c) prevention or reduction of intimal hyperplasia and/or restenosis (e.g., resulting from insertion of vascular grafts or hemodialysis access devices); and (d) may be used in affiliation with devices and implants that lead to scarring as described herein (e.g., as a sleeve or mesh around a breast implant to reduce or inhibit scarring).

The coated mesh may be applied to any bodily conduit or any tissue that may be prone to the development of fibrosis or intimal hyperplasia. Prior to implantation, the mesh may be trimmed or cut from a sheet of bulk material to match the configuration of the widened foramen, canal, or dissection region, or at a minimum, to overlay the exposed tissue area. The mesh may be bent or shaped to match the particular configuration of the placement region. The mesh may also be rolled in a cuff shape or cylindrical shape and placed around the exterior periphery of the desired tissue. The mesh may be provided in a relatively large bulk sheet and then cut into shapes to mold the particular structure and surface topography of the tissue or device to be wrapped. Alternatively, the mesh may be pre-shaped into one or more patterns for subsequent use. The films and meshes may be typically rectangular in shape and be placed at the desired location within the surgical site by direct surgical placement or by endoscopic techniques. The mesh may be secured into place by wrapping it onto itself (i.e., self-adhesive), or by securing it with sutures, staples, sealant, and the like. Alternatively, the mesh may adhere readily to tissue and therefore, additional securing mechanisms may not be required.

In certain embodiments, the present invention provides a method of reducing surgical adhesion comprising: placing a mesh coated with a polymeric coating composition on tissue, wherein the polymeric coating composition comprises a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In various embodiments, in terms of percentage, m is about 1040, about 15-35 or about 20-30.

In various embodiments, the X block is a poly(ethylene oxide) and the Y block is a polylactide (PDLLA). The X block may further comprise a terminal alkyl group, e.g., a methyl.

In certain embodiments, the diblock copolymers of the polymeric coating compositions are MePEG:PDLLA (with the ratios from 50:50 to 10:90), wherein the MePEG has a molecular weight of about 5000.

In one embodiment, the polymeric coating composition further comprises a therapeutic agent, such as an anti-fibrotic agent, as defined herein. In certain embodiments, the anti-fibrotic agent is present in the polymeric coating composition from about 0.01 to 8.0%, from about 0.5 to 5.5% or from about 1.0 to 10% of the total weight of the polymeric coating composition. In one embodiment, the anti-fibrotic agent is paclitaxel.

In one embodiment, the mesh of the present invention may be used to prevent or reduce adhesions that occur between tissues following surgery, injury or disease. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicate a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule, which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurological, pleural, thoracic and gynecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

In one embodiment, the mesh of the present invention may be used to prevent or reduce surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

In another embodiment, the mesh of the present invention may be used to prevent or reduce the fibrosis from occurring between a hernia repair mesh and the surrounding tissue. Hernias are abnormal protrusions (outpouchings) of an organ or other body structure through a defect or natural opening in a covering membrane, muscle or bone. Hernias themselves are not dangerous, but can become extremely problematic if they become incarcerated. Surgical prostheses used in hernia repair (referred to herein as “hernia meshes”) include prosthetic mesh-or gauze-like materials, which support the repaired hernia or other body structures during the healing process. Hernias are often repaired surgically to prevent complications. Conditions in which a hernia mesh may need to be used include, without limitation, the repair of inguinal (i.e., groin), umbilical, ventral, femoral, abdominal, diaphragmatic, epigastric, gastroesophageal, hiatal, intermuscular, mesenteric, paraperitoneal, rectovaginal, rectocecal, uterine, and vesical hernias. Hernia repair typically involves returning the viscera to its normal location and the defect in the wall is primarily closed with sutures, but for bigger gaps, a mesh is placed over the defect to close the hernia opening. Inclusion of an anti-scarring agent or composition comprising an anti-scarring agent into or onto a hernia repair mesh may reduce or prevent fibrosis proximate to the implanted hernia mesh, thereby minimizing the possibility of adhesions between the abdominal wall or other tissues and the mesh itself, and reducing further complications and abdominal pain.

In yet another embodiment, the mesh of the present invention may be used to prevent or reduce hypertrophic scars or keloids (e.g., resulting from tissue burns or other wounds). Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury, which is severe enough to break the skin. During this phase, which lasts 3 to 4 days, blood and tissue fluid form an adhesive coagulum and fibrinous network, which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. A mesh that comprises an anti-scarring agent or a composition that comprises an anti-scarring agent may be placed in contact with a wound or burn site in order to prevent formation of hypertrophic scar or keloids.

In yet another embodiment, the mesh of the present invention may be used for delivering an anti-scarring agent to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Examples of conditions that may be treated or prevented with fibrosis-inhibiting films and meshes include iatrogenic complications of arterial and venous catheterization, complications of vascular dissection, complications of gastrointestinal passageway rupture and dissection, restenotic complications associated with vascular surgery (e.g., bypass surgery), and intimal hyperplasia.

In one embodiment, the mesh of the present invention may be used to deliver an anti-fibrotic agent to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

The mesh (or film) of the present invention may be used in the form of a perivascular wrap to prevent restenosis at anastomotic sites resulting from insertion of vascular grafts or hemodialysis access devices. In this case, perivascular wraps may be incorporated with or coated with a fibrosis-inhibiting agent, which can be used in conjunction with a vascular graft to inhibit scarring at an anastomotic site. These films or meshes may be placed or wrapped in a perivascular (periadventitial) manner around the outside of the anastomosis at the time of surgery. The mesh implants comprising an anti-scarring agent may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

Regardless of the method of application of the drug to the mesh, the exemplary anti-fibrotic agents, used alone or in combination, should be administered under the following dosing guidelines. The total amount (dose) of anti-fibrotic agent in or on the mesh may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of mesh surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

2) Method of Using Injectable Microparticles Encaspulated With Polymeric Coating Compositions

The present invention provides a method of delivering microparticles to a host comprising: injecting a formulation comprising microparticles, the microparticles being encapsulated in a polymeric coating composition, wherein the polymeric coating composition comprises a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In various embodiments, in terms of percentage, m is about 40-75, about 45-70 or about 50-65.

In various embodiments, the X block is a poly(ethylene oxide) and the Y block is a polylactide (PDLLA). The X block may further comprise a terminal alkyl group, e.g., a methyl.

In certain embodiments, the diblock copolymers of the polymeric coating compositions are MePEG:PDLLA (with the ratios from 65:35 to 60:40), wherein the MePEG has a molecular weight of about 5,000.

In one embodiment, the microparticles are therapeutic agents that induce fibrotic tissue growth. For example, the microparticles are arterial wall irritants. Suitable microparticles include, but are not limited to: silk, talcum powder, chitosan copper, saracin, silica, crystalline silicates, quartz dust. In a preferred embodiment, the microparticles are silk powders.

In another embodiment, the polymeric coating composition further comprises a fibrosing agent, as defined herein. In one embodiment, the fibrosing agent is bleomycin.

In other embodiments, the formulation further comprises a buffer.

The injectable microparticle formulations can be delivered to a desirable location in a host, according to known methods in the art. More specifically, the encapsulated microparticles can be delivered at an interface between an implanted medical device (e.g., a stent graft) and the surrounding tissue, to immobilize or improve the adhesion of the medical device.

Also provided by the present invention are methods for treating patients having aneurysms (e.g., abdominal, thoracic, or iliac aortic aneurysms), for bypassing a diseased portion of a vessel, or for creating communication or a passageway between one vessel and another (e.g., artery to vein or vice versa, or artery to artery or vein to vein), such that risk of rupture of the aneurysm is reduced or prevented. In one embodiment, the method comprises injecting an injectable formulation comprising microparticles (e.g., silk) encapsulated in a polymeric coating composition. As utilized herein, it should be understood that “reduction in the risk of rupture” or “prevention of the risk of rupture” refers to a statistically significant reduction in the, number, timing, or, rate of rupture, and not to a permanent prohibition of any rupture.

In a more specific embodiment, the present invention provides a method of treating aneurysm comprising: injecting a formulation comprising silk particles to an aneurysm sac, each silk particle being encapsulated in a polymeric coating composition, wherein the polymeric coating composition comprises a bioerodable diblock copolymer of Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100.

In another embodiment, the polymeric coating composition further comprises a fibrosing agent, as defined herein. In one embodiment, the fibrosing agent is silk or bleomycin.

In other embodiments, the formulation further comprises a buffer.

The silk formulation may be injected into the aneurysm sac using, for example, a catheter, or using other means known to those skilled in the art to promote scarring of the aneurysm. In certain embodiments, the fibrosing agent or composition including the agent may be used in conjunction with a stent graft to repair an aneurysm.

Conventionally, injectable silk formulations encounter a number of drawbacks, which reduce the efficiency of the delivery. For example, blockage in the catheter during delivery may occur for formulations comprising silk fibers. Formulations comprising silk powder may experience back flux, whereby a portion of the injected silk powders leaks out of the injection site (e.g., a blood vessel) when the catheter (or needle) is withdrawn.

The silk formulation according to the present invention improves the efficiency of the delivery by preventing the back flux and/or diffusion of the silk powders. More specifically, the polymeric coating composition swells in the formulation, which forms a soft and deformable gel layer encasing the silk powders (either partially or fully). The encased silk powders passes through the catheter during injection and revert to the swollen state in the aneurysm sac. The back flux can therefore be effectively curbed according to the method described herein. Moreover, because the polymeric coating composition is bioerodable, the silk powders will be exposed in a period of the time to stimulate fibrosis.

The present invention is described in further detail by the following non-limiting examples. Examples

### EXAMPLE 1

### Polymerizing of MePEG-PDLLA-6535

65 g of methoxy polyethylene glycol (MePEG) with a molecular weight of 5,000 Dalton (Polysciences, Cat # 05986) were weighed in a 250 ml flat bottom (FB) flask. 35 g of D, L-lactide (Purasorb) were weighed separately in a weighing boat. Both MePEG5000 and D, L-lactide were dried under vacuum overnight at room temperature before use.

An oil-bath with light or heavy mineral oil (Aldrich, CAT# 33076-0) was heated to and maintained at 135° C. by using a thermo-controller (VWR, Model, LN: 002392, PN: 400188-REV A).

0.3-0.5 ml (Appr. 300-500 mg) of stannous 2-ethyl-hexanoate catalyst (Sigma, >95%, CAT# 33076-0) was added into the FB flask and then the flask was purged slowly with N₂ (oxygen free, Praxair, Grade 4.8) for 5 minutes.

The flask was stoppered with a glass stopper and placed into the oil-bath, and the magnetic stirrer was gradually turned on to a setting at 6 (Corning Thermo Stirrer/Hot Plate, Model PC-620). After 30 minutes, the flask was removed from the oil-bath. The D, L-lactide was added into the flask that was then purged slowly with oxygen free N₂ for 5 minutes. The flask was stoppered once again and was placed back into the oil-bath.

The magnetic stirrer was turned on to a setting of 3 and the polymerization reaction was continued for at least five (5) hours.

The FB-flask was removed from the oil bath. The molten polymer was poured into a glass container and was allowed to cool to room temperature.

The resulted diblock copolymer MePEG-PDLLA-6535 was labeled and stored in a refrigerator at 2-8° C.

### EXAMPLE 2

### Purification of MePEG-PDLLA-6535

To about 75 g of MePEG-PDLLA (65:35) in a 1000 ml flat-bottom titration and culture flask, isopropanol (HPLC grade) was added until it reached the 1000 ml mark.

The flask was placed in a 60° C. water bath (a 2000 ml jacket beaker connected with a VWR Isotemp Circulator, Model 1130-1) and the mixture was stirred till the MePEG-PDLLA (65:35) dissolved.

The solution was cooled down to room temperature (20-22° C.) to precipitate the diblock polymer, which was isolated by filtration. The precipitant was washed three times with 200-250 ml isopropanol each.

The polymer was first dried in the open air overnight for approximately 18 hours to remove most of the solvents. The pre-dried polymer was then transferred to a vacuum oven. The polymer was dried until the residual solvent was below the acceptable level (about 24 hours).

The dried polymer was stored in a refrigerator at 2-8° C. for use.

### EXAMPLE 3

### Coating of MePEG-PDLLA (20:80), 5% Paclitaxel on a Perivascular Wrap

The VICRYL or PLGA meshes (PolyMed) were cut into the size of 2×5 cm². The meshes were washed using HPLC grade isopropanol and completely dried in the forced-air oven at 50° C. The weight of each bare mesh was recorded.

About 1.9 g of MePEG-PDLLA-2080 was dissolved in 10 mL of acetone or dichloromethane (Calcdon, HPLC grade) to form a target of 20% solution.

About 100 mg of paclitaxel were added into each polymer solution. Paclitaxel was dissolved completely by placing the vials on Nutator Rotor (Model 421105, SN: 1100-15989).

The mesh was coated by dipped into the polymer/paclitaxel solution. The mesh was then removed from the vial while removing any excessive amount of solution on the mesh).

The coated mesh was dried 3-5 minutes in the air. The coated mesh was thereafter placed in a PTFE petri-disk, transferred into a vacuum oven, and continued drying under vacuum overnight at room temperature.

The dried samples were weighed and packed between two pieces of release-liners (REXAM A10, Grade 10393, silicone coated PET) and sealed in a Pouche bag.

### EXAMPLE 4

### Preparation of MePEG-PDLLA-6535 Encapsulated Silk Microparticles

2600 mg of MePEG-PDLLA (65:35) and 2400 mg of silk (degummed or virgin) particles (with a size distribution of 10-100, 100-500 or 500-1000 μm; actual size depending on need) were mixed in 200 mL of dichloromethane (DCM). The silk particles were suspended in MePEG-PDLLA-6535/DCM solution. The suspension was spray-dried with a Buchi mini Spray-dryer (Model: B-191) under following conditions: Aspirator rate: 100%; Nitrogen flow rate: 600 L/h; Pressure: 6 bars; Inlet temp.: <50° C.; and Outlet temp.: <37° C.

All the MePEG-PDLLA (65:35)-encapsulated silk microparticles were collected and dried under vacuum until the remained dichloromethane level below expectation. They were stored at 2-8° C. for use.

### EXAMPLE 5

### Injectable Microparticle Formulation for Treating Abdominal Aortic Aneurysm (AAA)

The MePEG-PDLLA (65:35)-encapsulated silk microparticles, as prepared according to Example 4, were weighed out in a syringe. The encapsulated silk microparticles were mixed with a pH 7.3 buffer from another syringe. MePEG-PDLLA (65:35) layer on the outside of the silk microparticles started to adsorb water and swell to form a gel layer outside of the silk particles. The gel layer was soft and allowed the silk particles to be injected through a small needle or catheter. After injection, the silk particles were locked in the site of application without leaking out to the blood vessel during and after pulling out of the catheter, due to significant increase of the sizes of the encapsulated silk microparticles. The MePEG-PDLLA (65:35) layer started to erode in a few days after injection, physically exposed the silk particles to stimulate the tissue growth.

### EXAMPLE 6

### Coating Formulations

Five different MePEGIPLLA coating formulations were prepared to compare the effect of various ratios of PEG to block copolymer and the different solvent mixtures on solubility, clarity, and stability. Such formulations may be used to coat a variety of medical devices, as described herein. A dye (e.g., gentian violet or dimethylene blue) was incorporated into the formulations, since dyes are frequently used to aid in visual examination of coating uniformity once formulations have been coated onto a substrate.

Formulation A

| | | |
|---|---|---|
| Ethanol | 6.00 | g |
| Deionized water | 0.50 | g |
| Acetonitrile | 5.71 | g |
| PEG (20K) | 3.00 | g |
| PEG/PLLA 60:40 | 0.39 | g |
| Gentian Violet solution | 4 | drops |

Formulation B

| | | |
|---|---|---|
| Ethanol | 6.01 | g |
| Acetonitrile | 5.70 | g |
| PEG (20K) | 3.02 | g |
| PEG/PLLA 60:40 | 0.53 | g |
| Gentian Violet solution | 4 | drops |

Formulation C

| | |
|---|---|
| Ethanol | 6.00 g |
| Acetonitrile | 5.74 g |
| PEG (20K) | 3.01 g |
| PEG/PLLA 60:40 | 0.65 g |
| Dimethylmethylene blue | trace |

Formulation D

| | |
|---|---|
| Isopropanol | 7.51 g |
| Deionized water | 2.93 g |
| PEG (20K) | 3.0 g |
| PEG/PLLA (60:40) | 0.65 g |
| 1% aq. TWEEN 80 | 0.07 g |
| Gentian Violet solution | 0.11 g |

Formulation E

| | |
|---|---|
| Isopropanol | 7.51 g |
| Deionized water | 2.92 g |
| PEG (20K) | 3.02 g |
| PEG/PLLA (60:40) | 1.02 g |
| 1% aq. TWEEN 80 | 0.09 g |
| Gentian Violet solution | 0.10 g |

Formulations A-D (3:0.39 to 3:0.65 ratio of PEG to diblock copolymer) yielded clear, homogenous solutions. Formulations having PEG to diblock copolymer ratios of about 3:0.6 to about 3:0.8 also would likely form clear, homogeneous, stable solutions. Although Formulations C and D had different solvent mixtures, both formed clear, stable solutions, suggesting that solubility of diblock copolymer in the mixture was not dependent on choice of solvent. Formulation E, in comparison, having a 3:1 ratio of PEG to diblock copolymer did not completely dissolve and formed a hazy mixture.

### EXAMPLE 7

### Testing of Needle Coating Formulation

Insulin pump needles (MINIMED bent needles) were coated and tested in vivo with Formulation F according to the following procedure. The tests disclosed in this example were conducted in one human subject, the inventor.

Formulation F

| | |
|---|---|
| Ethanol | 6.01 g |
| Acetonitrile | 5.75 g |
| 5-fluorouracil | 0.09002 g |
| PEG (20K) | 3.00 g |
| PEG/PLLA (60:40) | 0.49 g |

The needles were connected to a delivery tube that was connected to a MINIMED 715 insulin pump. The needles were coated with the needle in a vertical orientation, with the tip down. A needle was dipped into the coating liquid, and withdrawn at the same orientation at a rate of 4-5 cm/second. Care was taken to avoid coating the needle lumen. The needle was maintained in a vertical orientation and dried at room temperature for 3 minutes and then at 50° C. for 3 minutes using a hairdryer at a distance of about 5-8 cm from the needle surface. Coated needles were inserted through an anti-microbial cuff and then inserted transcutaneously into the patient. The cuffs used in the procedure were coated with an anti-infective benzalkonium chloride hybrid polymer coating as described in U.S. Pat. No. 6,368,611. The pump used a 3 ml syringe reservoir that was filled with NOVALOG U-100 insulin. The insulin pump had the basal rate set at 1.2 units per hour from 4:00 am to 9:00 am, followed by 0.9 units per hour from 9:00 am to 12 noon, followed by 0.7 units per hour from noon till 12:00 am, and at 0.6 units per hour from 12:00 am to 4:00 am. This basal rate produced declining, fasting blood glucose levels in the mornings for a few days after the needle was first inserted into subcutaneous fatty tissue of the abdominal region. After two to four days, the declining, fasting blood sugar ceased. This effect was thought to be due to protein absorption around the distal portion of the needle, which protein absorption appears to interfere with the absorption of the insulin into the surrounding tissue. No infections were noted during any of the following insertion trials with the coated needles. Needles were inserted, and blood glucose levels were recorded on the order of 10-16 times per day. The needles were removed when the fasting blood glucose levels stopped declining. When the fasting blood glucose levels stopped declining, they would typically begin to ascend, rather than exhibit a plateau behavior. After removal, the days of implantation were recorded. Needles coated with the Formulation F were tested in four rounds in vivo as described and showed an average patency time of 6.5 days.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. The invention having been described, the following examples are intended to illustrate, and not limit, the invention.

1. A device comprising:
an insertable medical device; and
a polymeric coating composition comprising a bioerodable diblock copolymer of Formula: X—Y (m:n) having a molecular weight of at least 7,500, wherein,
X is a hydrophilic poly(alkylene oxide) having a molecular weight of at least 3,500,
Y is a hydrophobic polyester,
m represents a weight percentage of X based on a total weight of the diblock copolymer,
n represents a weight percentage of Y based on the total weight of the diblock copolymer, and
m+n=100.

2. The device of claim 1 wherein X is poly(ethylene oxide).

3. The device of claim 2 wherein X further comprises a terminal alkyl group.

4. The device of claim 3 wherein the terminal alkyl group is methyl or ethyl.

5. The device of claim 4 wherein X is methyl polyethylene glycol (MePEG).

6. The device of claim 5 wherein X has a molecular weight of about 5000.

7. The device of claim 1 wherein Y comprises residues of a hydroxy acid.

8. The device of claim 7 wherein Y comprises residues of lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one or 1,5-dioxepan-2-one.

9. The device of claim 8 wherein Y is polylactide (PDLLA).

10. The device of claim 1 wherein the diblock copolymer comprises MePEG and PDLLA.

11. The device of claim 1 wherein m:n is about 65:35, 60:40, 50:50, 45:55, 40:60, 35;65, 30:70, 25:75, 20:80, 15:85 or 10:90.

12. The device of claim 1 wherein the polymeric coating composition further comprises a therapeutic agent.

13. The device of claim 12 wherein the therapeutic agent is an anti-infective agent, an anti-fibrotic agent, an anticancer agent, an anti-inflammatory agent or a combination thereof.

14. The device of claim 13 wherein the anti-infective agent is selected from 2-bromo-2-nitropropane-1,3-diol (BRONOPOL), Irgasan (TRICLOSAN), polyhexanide (VANTOCIL IB, COSMOCIL CQ, or BAQUACIL), benzalkonium chloride, benzethonium chloride, cetylpyradinium chloride, stearalkonium chloride, phenol, cresol, aminophenol, iodine, iodide, 8-hydroxyquinolone, chlorhexidine, anthracyclines, fluoropyrimidines, folic acid antagonists, podophylotoxins, camptothecins, hydroxyureas, and platinum complexes.

15. The device of claim 13 wherein the anti-fibrotic agent is paclitaxel, rapamycin, everolimus, zotarolimus, chlorpromazine, or mycophenolic acid.

16. The device of claim 12 wherein the therapeutic agent is a fibrosing agent.

17. The device of claim 16 wherein the fibrosing agent is silk.

18. The device of claim 1 wherein the insertable medical device is a needle or catheter.

19. The device of claim 18 wherein X comprises residues of ethylene oxide, and Y comprises residues of lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one or 1,5-dioxepan-2-one.

20. The device of claim 19 wherein X is MePEG and Y is polylactide.

21. The device of claim 19 wherein the diblock copolymer is MePEG-PDLLA (60:40) and MePEG has a molecular weight of about 5000.

22. The device of claim 19 wherein the polymeric coating composition further comprises a second polymer.

23. The device of claim 22 wherein the second polymer is polyethylene glycol (PEG).

24. The device of claim 23 wherein the PEG has a molecular weight of 200, 300, 400, 1,000, 1,450, 1,500, 2,000, 3,000, 3,350, 4,000, 6,000, 8,000,10,000, 20,000, and 35,000.

25. The device of claim 22 wherein a weight ratio of the diblock copolymer to the PEG is between about 1:9 and 1:3.

26. The device of claim 25 wherein the weight ratio of the diblock copolymer to the PEG is 1:5.

27. The device of claim 18 wherein the polymeric coating composition further comprises an anti-infective agent.

28. The device of claim 27 wherein the polymeric coating composition comprises about 0.1% to 50% of the anti-infective agent.

29. The device of claim 27, wherein the polymeric coating composition comprises about 0.5% to 30% of the anti-infective agent.

30. The device of claim 27, wherein the polymeric coating composition comprises about 3% to 20% of the anti-infective agent.

31. The device of claim 18 wherein the polymeric coating composition further comprises an anti-fibrotic agent.

32. The device of claim 31 wherein the polymeric coating composition comprises about 0.01% to 8.0% of the anti-fibrotic agent.

33. The device of claim 31, wherein the polymeric coating composition comprises about 0.5% to 5.5% of the anti-fibrotic agent.

34. The device of claim 31, wherein the polymeric coating composition comprises about 0.5% of the anti-fibrotic agent.

35. The device of claim 31 wherein the anti-fibrotic agent is paclitaxel.

36. The device of claim 1 wherein the insertable medical device is a mesh.

37. The device of claim 36 wherein the mesh is formed of bioerodable material.

38. The device of claim 36 wherein the mesh is formed of a non-bioerodable material.

39. The device of claim 36 wherein X comprises residues of ethylene oxide, and Y comprises residues of lactide, lactic acid (both D and L forms), glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one, or 1,5-dioxepan-2-one.

40. The device of claim 39 wherein X is MePEG and Y is polylactide.

41. The device of claim 40 wherein the diblock copolymer is MePEG-PDLLA (50:50), or MePEG-PDLLA (45:55), or MePEG-PDLLA (40:60), or MePEG-PDLLA (35:65), or MePEG-PDLLA (30:70), or MePEG-PDLLA (25:75), or MePEG-PDLLA (20:80), or MePEG-PDLLA (15:85), or MePEG-PDLLA (10:90) and MePEG has a molecular weight of about 5,000.

42. The device of claim 39 wherein the polymeric coating composition further comprises a therapeutic agent.

43. The device of claim 42 wherein the therapeutic agent is an anti-fibrotic agent, anticancer agent, an anti-infective agent, an anti-inflammatory or a combination thereof.

44. The device of claim 43 wherein the therapeutic agent is paclitaxel.

45. The device of claim 42 wherein the therapeutic agent is a fibrosing agent.

46. The device of claim 39 wherein the polymeric coating composition comprises about 2% to 25% of the therapeutic agent.

47. The device of claim 39 wherein the polymeric coating composition comprises about 5% to 20% of the therapeutic agent.

48. The device of claim 39 wherein the polymeric coating composition comprises about 8% tol 5% of the therapeutic agent.

49. The device of claim 1 wherein the insertable medical device is an injectable formulation comprising microparticles, and the microparticles are encapsulated in the polymeric coating composition.

50. The device of claim 49 wherein the microparticles are silk powders.

51. The device of claim 49 wherein X comprises residues of ethylene oxide, and Y comprises residues of lactide, lactic acid (both D and L forms), glycolide, glycolic acid, α-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one or 1,5-dioxepan-2-one.

52. The device of claim 51 wherein the polymeric coating composition comprises a diblock copolymer MePEG-PDLLA (65:35), or MePEG-PDLLA (60:40), MePEG having a molecular weight of about 5,000.

53. The device of claim 49 wherein the polymeric coating composition further comprises a fibrosing agent.

54. The device of claim 49 wherein the injectable formulation further comprises a buffer.

55. The device of claim 49 wherein the injectable formulation further comprises a second polymer.

56. The device of claim 55 wherein the second polymer is COSEAL®.

57. A method of preparing an insertable medical device comprising: coating the insertable medical device with a polymeric coating composition comprising a bioerodable diblock copolymer of Formula: X—Y (m:n) having a molecular weight of at least 7,500, wherein,
X is a hydrophilic poly(alkylene oxide) having a molecular weight of at least 3,500,
Y is a hydrophobic polyester,
m represents a weight percentage of X based on a total weight of the diblock copolymer,
n represents a weight percentage of Y based on the total weight of the diblock copolymer, and
m+n=100.

58. The method of claim 57 further comprising, prior to coating, preparing a pre-coating solution of the polymeric coating composition in a solvent.

59. The method of claim 57 further comprising mixing a therapeutic agent in the pre-coating solution.

60. The method of claim 59 wherein the therapeutic agent is an anti-fibrotic agent, an anti-infective agent, an anticancer agent, anti-inflammatory agent, or a combination thereof.

61. The method of claim 59 wherein the therapeutic agent is a fibrosing agent.

62. The method of claim 58 further comprising mixing a second polymer in the pre-coating solution.

63. The method of claim 62 wherein the second polymer is PEG.

64. The method of claim 57 further comprising, after coating, removing the solvent.

65. The method of claim 57 wherein coating comprises dipping or spraying a surface of the insertable medical device.

66. The method of claim 57 wherein the insertable medical device is a needle or catheter.

67. The method of claim 66 wherein the polymeric coating composition comprises: MePEG-PDLLA (60:40), MePEG having a molecular weight of about 5000, PEG, and a therapeutic agent.

68. The method of claim 67 wherein the therapeutic agent is an anti-infective agent, an anti-fibrotic agent, an anticancer agent, an anti-inflammatory agent or a combination thereof.

69. The method of claim 57 wherein the insertable medical device is a mesh.

70. The method of claim 69 wherein the polymeric coating composition comprises: MePEG-PDLLA (50:50), or MePEG-PDLLA (45:55), or MePEG-PDLLA (40:60), or MePEG-PDLLA (35:65), or MePEG-PDLLA (30:70), or MePEG-PDLLA (25:75), or MePEG-PDLLA (20:80), or MePEG-PDLLA (15:85), or MePEG-PDLLA (10:90), MePEG having a molecular weight of about 5,000, and a therapeutic agent.

71. The method of claim 70 wherein the therapeutic agent is an anti-infective agent, an anti-fibrotic agent, an anticancer agent, an anti-inflammatory agent, or a combination thereof.

72. The method of claim 57 wherein the insertable medical device is an injectable formulation comprising microparticles.

73. The method of claim 72 wherein the microparticles are silk.

74. The method of claim 72 wherein the polymeric coating composition comprises MePEG-PDLLA (65:35) or MePEG-PDLLA (60:40), MePEG having a molecular weight of about 5000.

75. The method of claim 74 wherein the polymeric coating composition further comprises a second polymer.

76. The method of claim 75 wherein the polymer is COSEAL®.

77. A method of reducing surgical adhesion comprising: placing a mesh coated with a polymeric coating composition at a surgical site of a host, the polymeric coating composition comprising a bioerodable diblock copolymer of Formula: X—Y (m:n) having a molecular weight of at least 7,500, wherein,
X is a hydrophilic poly(alkylene oxide) having a molecular weight of at least 3,500,
Y is a hydrophobic polyester,
m represents a weight percentage of X based on a total weight of the diblock copolymer,
n represents a weight percentage of Y based on the total weight of the diblock copolymer, and
m+n=100.

78. The method of claim 77 wherein the polymeric coating composition comprises: MePEG-PDLLA (50:50), or MePEG-PDLLA (45:55), or MePEG-PDLLA (40:60), or MePEG-PDLLA (35:65), or MePEG-PDLLA (30:70), or MePEG-PDLLA (25:75), or MePEG-PDLLA (20:80), or MePEG-PDLLA (15:85), or MePEG-PDLLA (10:90), MePEG having a molecular weight of about 5,000, and a therapeutic agent.

79. The method of claim 77 wherein the therapeutic agent is an anti-infective agent, an anti-fibrotic agent, an anticancer agent, an anti-inflammatory agent, or a combination thereof.

80. A method of treating aneurysm comprising: delivering an injectable formulation comprising microparticles to an aneurysm sac, the microparticles being coated with a polymeric coating composition comprising a bioerodable diblock copolymer of Formula: X—Y (m:n) having a molecular weight of at least 7,500, wherein,
X is a hydrophilic poly(alkylene oxide) having a molecular weight of at least 3,500,
Y is a hydrophobic polyester,
m represents a weight percentage of X based on a total weight of the diblock copolymer,
n represents a weight percentage of Y based on the total weight of the diblock copolymer, and
m+n=100.

81. The method of claim 80 wherein the microparticles are silk.

82. The method of claim 80 wherein the diblock copolymer is MePEG-PDLLA (65:35) or MePEG-PDLLA (60:40).

83. The method of claim 80 wherein the polymeric coating composition further comprises a buffer.

84. The method of claim 80 wherein the polymeric coating composition may further comprise a fibrosing agent.

85. A method of preparing an injectable formulation having microparticles comprising:
mixing microparticles and a diblock copolymer in a solvent to provide a suspension, the diblock copolymer being represented by Formula: X—Y (m:n) having a molecular weight of at least 7,500, wherein, X is a hydrophilic poly(alkylene oxide) having a molecular weight of at least 3,500, Y is a hydrophobic polyester, m represents a weight percentage of X based on a total weight of the diblock copolymer, n represents a weight percentage of Y based on the total weight of the diblock copolymer, and m+n=100; and
spray-drying the suspension to provided diblock copolymer-coated microparticles.

86. The method of claim 85 wherein the microparticles are silk.

87. The method of claim 85 wherein the polymeric coating composition comprises MePEG-PDLLA (65:35) or MePEG-PDLLA (60:40), MePEG having a molecular weight of about 5000.

88. The method of claim 85 wherein the polymeric coating composition further comprises a second polymer.

89. The method of claim 88 wherein the second polymer is COSEAL®.

90. The method of claim 85 further comprising mixing a fibrosing agent in the suspension.

91. A method of extending the patency of an insertable medical device comprising coating the insertable medical device with a polymeric coating composition comprising a bioerodable diblock copolymer of Formula: X—Y (m:n) having a molecular weight of at least 7,500, wherein,
X is a hydrophilic poly(alkylene oxide) having a molecular weight of at least 3,500,
Y is a hydrophobic polyester,
m represents a weight percentage of X based on a total weight of the diblock copolymer,
n represents a weight percentage of Y based on the total weight of the diblock copolymer, and
m+n=100.

92. The method of claim 91 wherein the polymeric coating composition comprises MePEG-PDLLA (60:40), MePEG having a molecular weight of about 5,000, PEG, and a therapeutic agent.

93. The method of claim 92 wherein the therapeutic agent is an anti-infective agent, an anti-fibrotic agent, an anticancer agent, an anti-inflammatory agent, or a combination thereof.

94. The method of claim 91 wherein the insertable medical device remains its patency for 1 day, 2 days, 3 days, 4 days, 5 day, 6 days or 7 days.
